SEQUENCE LISTING (4 sequences)

SEQ ID NO: 1 - DNA, 18 bp, Artificial Sequence
  Forward primer of Examples 1 and 3
gacggaccagagcgaaag

SEQ ID NO: 2 - DNA, 19 bp, Artificial Sequence
  Reverse primer of Examples 1 and 3
cgtcttcgaacctccgact

SEQ ID NO: 3 - DNA, 21 bp, Artificial Sequence
  Forward primer of Example 2
cccaaacctgtaaaaccagtg

SEQ ID NO: 4 - DNA, 21 bp, Artificial Sequence
  Reverse primer of Example 2
ttgtgaataggtgccagtgaa

(19) United States
(12) Patent Application Publication
(10) Pub. No.: US 2012/0225428 A1
(43) Pub. Date: Sep. 6, 2012

(54) TYPE OF UNIVERSAL PROBE FOR THE DETECTION OF GENOMIC VARIANTS

(75) Inventors: Reinhard Beck, Penzberg (DE); Frank Bergmann, Iffeldorf (DE); Rita Haerteis, Ried-Kochel a. S. (DE); Dieter Heindl, Paehl (DE); Ralf Mauritz, Penzberg (DE); Heiko Walch, Muenchen (DE)

(73) Assignee: ROCHE DIAGNOSTICS OPERATIONS, INC., Indianapolis, IN (US)

(21) Appl. No.: 13/411,902

(22) Filed: Mar. 5, 2012

(30) Foreign Application Priority Data
Mar. 4, 2011 (EP) 11001840.5

(51) Int. Cl. C12Q 1/68; G01N 21/64

(52) U.S. Cl. 435/6.11

(57) ABSTRACT

The present disclosure relates to a composition comprising a first set of probes and a second set of probes, composed of one or more DNA nucleotide(s) and five or more LNA (locked nucleic acid) nucleotides, wherein the base at a discriminating position differs for a first probe of the first set and a first probe of the second set. The present disclosure relates to the composition comprising a plurality of probes in each of the first and second set of probes, wherein the probes in each set differ in one, two, or three LNA random position(s). Further, the present disclosure relates to a method of detecting genomic variants by means of the aforementioned probes.

### PRIORITY CLAIM

This application claims the benefit of European Patent Application No 11001840.5, filed Mar. 4, 2011, the disclosure of which is hereby incorporated by reference in its entirety.

### SEQUENCE LISTING

The instant application contains a Sequence Listing which has been submitted in ASCII format via EFS-Web and is hereby incorporated by reference in its entirety. Said ASCII copy, created on Mar. 4, 2012, is named SEQUENCE_LISTING_{—}27180US.txt, and is 1,051 bytes in size.

### FIELD OF THE DISCLOSURE

The present disclosure relates to a method of detecting genomic variants. More specifically, the present disclosure relates to a composition for detecting genomic variants, the composition comprising a first set of probes and a second set of probes, composed of one or more DNA nucleotide(s) and five or more LNA (locked nucleic acid) nucleotides (or LNA analogues such as ENA (2′-O,4′-C-ethylene-bridges nucleic acid) or 2′-amino-LNA derivatives), wherein the probes differ in one, two or three LNA random position(s) and the base at a discriminating position. The instant disclosure also relates to a library of a plurality of probes.

### BACKGROUND OF THE DISCLOSURE

Genetic polymorphisms play a role in the health and the predisposition of various diseases in nearly all organisms. Genetic polymorphisms and mutations may have a phenotypic impact on the affected organism. For example, a polymorphism may affect certain metabolic characteristics of the affected organism. It is also possible the polymorphism will remain unnoticed (e.g., comprise a silent mutation) if a non-coding part of the genome is affected, for example. In general, genetic polymorphisms are a mutation in a nucleotide of a gene. These mutations may comprise frameshift mutations, a deletion of a gene or a part of the gene, the repetition of a gene, the insertion of a gene or a single nucleotide exchange.

The deletion of a gene or a part thereof and the alteration in the copy numbers of a gene leads to a copy number variant (CNV). CNVs can be caused by genomic rearrangements such as deletions, duplications, and translocations. CNVs are often found in the connection with different types of cancer, such as, e.g., non-small cell lung cancer, and may also be found in connection with autism and schizophrenia.

A single nucleotide exchange leads to a single nucleotide polymorphism (SNP). Numerous diseases are associated with SNPs, such as sickle-cell anemia, hypercoagulability disorder associated with the variant Factor V Leiden, asthma predisposition, and cancer (e.g., oncogenes).

Consequently, analytic tools for detecting mutations, such as CNV or SNPs, in biological samples have impacted today's research and medicine. Additionally, in the view of a population that is more and more aged and wherein health care and health protection has an increasing importance, the detection of a genetic predisposition or a genetic disease becomes even more important. Further, the (still novel) field of personalized medicine is heavily based on the detection of genetic polymorphisms.

Several methods for detection of single nucleotide polymorphisms (SNPs) and copy number variants (CNVs) have been developed. For instance, genetic polymorphisms may be detected by methods such as, sequencing of the gene of interest, single-base extension (SBE), deoxyribonucleic acid (DNA) microarrays (e.g., a SNP array or an Affymetrix™ microarray chip), PCR based methods (for example, TaqMan® probe methods), array comparative genomic hybridization, or comparative in situ hybridization.

However, for all of the above methods, the synthesis of multiple highly specific probes is required. For each potential locus of a polymorphism or a mutation, at least two highly specific probes representing two different genotypes have to be synthesized and compared with another. Consequently, for each allele in question, a probe has to be individually synthesized. The analysis of numerous potential polymorphisms loci in one experiment is therefore highly time-consuming, laborious and costly. Additionally, for some of the above methods, each probe must also be labeled. This labeling procedure is again time-consuming, laborious and costly. Other methods, such as the array-based methods, provide thousands of different variants, but are hard to analyze and do not give quantitative results. Moreover, these methods fail to detect unknown mutations. In order to achieve high specificity, the probes must have a certain minimal length. Consequently, probes with a high specificity are comparably long and are therefore difficult to synthesize. Moreover, such long probes may fail to detect single nucleotide polymorphisms (SNPs) as the rational differences of a full match (e.g., fully complementary) and a single mismatch are too small.

On the other hand, there are methods for the quantification of expression levels. For instance, a polymerase chain reaction (PCR) may be performed on a real-time PCR cycler such as, e.g., a LightCycler®. The PCR can also be a reverse transcriptase PCR. Further, the PCR performed on a real-time PCR cycler can be combined with intercalating DNA dyes, such as SYBR Green I, or with a labeled probe, such as a specific or semi-specific probean exemplary semi-specific probe is the universal probe Iibrary™ (UPL) probe available from Roche Applied Science, Indianapolis, Ind., USA). However, these methods are, in some instances, unable to detect polymorphisms or mutations and are generally unable to quantify such mutations.

### SUMMARY OF THE DISCLOSURE

The present disclosure provides a set of probes comprising DNA and LNA nucleotides. According to embodiments of the preset disclosure, at the 5′ end of the probes, the nucleobases are determined, whereas at the 3′ end, there are one or more (for example, two or three) random nucleotides (also referred to herein as “wobble” positions). This probe design enables that a manageable number of probes may be applicable to a multitude of problems.

According to the instant disclosure, an embodiment consists of a short nucleic acid strand that can be used universally for the detection of various target sequences. The short nucleic acid sequence of the instant disclosure is also allele specific and enables the detection of a specific mutation, such as a single nucleotide polymorphism (SNP).

According to the present disclosure, some embodiments include a composition comprising a first probe and a second probe. According to such embodiments, the first probe has a 5′ end opposite a 3′ end and at least eight nucleotides, the at least eight nucleotides comprising at least one DNA nucleotide and at least five locked nucleic acid nucleotides and a first discriminating position; and a second probe having a 5′ end opposite a 3′ end and a same number of nucleotides as the first probe. The nucleotides of the second probe comprise a same number of DNA nucleotides and locked nucleic acid nucleotides as the first probe and a second discriminating position located at a position corresponding to the first discriminating position in the first probe. Also, according to such embodiments, the nucleotides of the first and second probes comprise one of an adenine nucleobase, a cytosine nucleobase, a guanine nucleobase, a thymine nucleobase, a uracil nucleobase, and a methyl cytosine nucleobase, and the first and second probes comprise differing nucleobases at the first and second discriminating positions. However, according to such embodiments, the first and second probes comprise the same nucleobases at all other nucleotide positions of the probes.

Other embodiments of the instant disclosure comprise a composition including a first and second set of probes. Each probe of the first and second sets have a 5′ end opposite a 3′ end and eight nucleotides. The nucleotides of each probe of the first set have at least one DNA nucleotide, at least five locked nucleic acid nucleotides, and a first discriminatory position, at least one locked nucleic acid nucleotide being a random locked nucleic acid nucleotide, whereas each probe of the second set of probes have a corresponding number of DNA nucleotides, locked nucleic acid nucleotides, and random locked nucleic acid nucleotides as a probe in the first set, and each probe of the second set has a second discriminating position located at a same nucleotide location as a first discriminating position of a probe in the first set. Also, according to some such embodiments, all probes of the first and second sets have a same nucleobase sequences with the exception of (i) the nucleobase at the random locked nucleic acid nucleotides; and (ii) the nucleobase at the first and second discriminating positions. Also, the nucleobase of the second discriminating position differs from the nucleobase of the first discriminating position at the same nucleotide location, and the at least one random locked nucleic acid nucleotide of each probe of the second set comprises a same nucleobase located at a same nucleotide location of the at least one random locked nucleic acid nucleotide of a probe of the first set. According to such embodiments, the nucleobase of the random locked nucleic acid nucleotide is selected from one of adenine, cytosine, guanine, and thymine, and any possible nucleobase sequence resulting from nucleobase variations at the one or more random locked nucleic acid nucleobase position(s) is represented by at least one probe in both the first and second set of probes.

According to other embodiments of the instant disclosure, a method of determining a genotype at a locus of interest in a sample comprising genetic material is provided. The method includes the steps of contacting the genetic material with a first probe and a second probe and detecting the binding of the first or second probe to the genetic material, thereby determining the genotype at the locus. According to such embodiments, the first and second probes each have a 5′ end opposite a 3′ end and eight nucleotides comprising at least one DNA nucleotide and at least five locked nucleic acid nucleotides. The nucleotides of the first probe comprise a first discriminating position and the nucleotides of the second probe comprise a second discriminating position at a same nucleotide location in the second probe as the first discriminating position in the first probe. Also, the first discriminating position comprises a different nucleobase than the second discriminating position, wherein the nucleobases at the other nucleotides of the first and second probes are the same.

Yet further embodiments of the instant disclosure include a composition including a first set of probes and a second set of probes, each of the probes having eight nucleotides being composed of one to three DNA nucleotides and five to seven LNA (locked nucleic acid) nucleotides. According to such embodiments, all probes of the first and the second set of probes have identical nucleotide sequences with the exception of (i) the base(s) at one, two or three LNA random position(s); and (ii) the base at a discriminating position, wherein the one, two or three LNA random position(s) and the discriminating position are located at identical positions in all probes of the first and the second set. Further, according to such embodiments at each LNA random position the base is independently selected from adenine, cytosine, guanine and thymine and any possible sequence resulting from the base variation(s) at the one, two or three LNA random position(s) is represented by at least one probe in each set of probes. Additionally, according to such embodiments, the base at the discriminating position is identical within each set of probes, but differs between the first and the second set of probes.

Some embodiments of the instant disclosure include a library of at least two sets of probes. According to such embodiments the library comprises a plurality of sets of probes each of the probes having eight nucleotides with the general structure 5′-D-L-L-L-L-L-X-X-3′ or 5′-D-L-L-L-L-X-X-X-3′ (where D is a DNA nucleotide; each L is a LNA nucleotide; and each X is a LNA random nucleotide). Also, within one set of probes, all probes have identical nucleotide sequences with the exception of the two and/or three LNA random nucleotides (with each position of a LNA random nucleotide base being independently selected from adenine, cytosine, guanine and thymine). Also, according to such embodiments, any possible sequence resulting from the base variation(s) at the two positions is represented by a probe in each set of probes and one set of probes differing from the other set of probes in the sequence of at least the DNA nucleotide D or an LNA nucleotide L.

According to yet another embodiment of the instant disclosure, a method of determining the genotype at a locus of interest in a sample obtained from a subject is provided. The method includes the steps of contacting the sample comprising the genetic material with any of the compositions of any of the composition embodiments provided herein, and detecting the binding of a probe of the first or the second set of probes to the genetic material, thereby determining the genotype at the locus.

### BRIEF DESCRIPTION OF THE DRAWINGS

The features of this disclosure, and the manner of attaining them, will become more apparent and the disclosure itself will be better understood by reference to the following description of embodiments of the disclosure taken in conjunction with the accompanying drawing.

FIG. 1 is depicts an embodiment of a PCR Dual Color Assay scheme with two hydrolysis probes according to the instant disclosure.

FIG. 2 depicts amplification curves of an embodiment of a mono color PCR assay with 18S parameter according to the instant disclosure.

FIG. 3 depicts amplification curves of an embodiment of a mono color PCR assay with MNAT1 parameter according to the instant disclosure.

FIG. 4 depicts amplification curves of an embodiment of a dual color PCR assay with 18S parameter according to the instant disclosure.

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplifications set out herein illustrate an exemplary embodiment of the disclosure, in one form, and such exemplifications are not to be construed as limiting the scope of the disclosure in any manner.

### BRIEF DESCRIPTION OF THE SEQUENCE LISTING

SEQ ID NO.: 1 is the nucleotide sequence for the forward primer of Example 1 and Example 3.

SEQ ID NO.: 2 is the nucleotide sequence for the reverse primer of Example 1 and Example 3.

SEQ ID NO.: 3 is the nucleotide sequence for the forward primer of Example 2.

SEQ ID NO.: 4 is the nucleotide sequence for the reverse primer of Example 2.

Although the sequence listing represents an embodiment of the present disclosure, the sequence listing is not to be construed as limiting the scope of the disclosure in any manner and may be modified in any manner as consistent with the instant disclosure and as set forth herein.

### DETAILED DESCRIPTION OF THE EMBODIMENTS OF THE DISCLOSURE

The embodiments disclosed herein are not intended to be exhaustive or limit the disclosure to the precise form disclosed in the following detailed description. Rather, the embodiments are chosen and described so that others skilled in the art may utilize their teachings.

In the context of the present disclosure, a set of probes comprising DNA and locked nucleic acid (“LNA”) nucleotides is provided. According to an embodiment of the instant disclosure, at the 5′ end, the nucleobases are determined, whereas at the 3′ end, there are one or more, preferably two or three, random nucleotides (wobble positions). This probe design enables that a manageable number of probes is sufficient to be applicable to a multitude of problems.

Surprisingly and unexpectedly, when using the set of probes as defined herein, the probes binding to specific targets are able to discriminate different alleles of specific mutations, for example single nucleotide polymorphisms (“SNPs”). Concomitantly, each set of probes can surprisingly be used for detecting SNPs of multiple different target genes.

An embodiment of the present disclosure relates to a composition comprising a first set of probes and a second set of probes. According to some embodiments, each of the probes (of the first and second set of probes) haseight nucleotides. In some embodiments, the eight nucleotides comprise one to three DNA nucleotides and five to seven LNA (locked nucleic acid) nucleotides. Further, according to some embodiments, all probes of the first and the second set of probes have identical nucleotide sequences with the exception of:
(i) base(s) at one, two or three LNA random position(s); and
(ii) a base at a discriminating position,
wherein the one, two or three LNA random position(s) and the discriminating position are located at identical positions in all probes of the first and the second set;
wherein at each LNA random position the base is independently selected from adenine, cytosine, guanine and thymine and any possible sequence resulting from the base variation(s) at the one, two or three LNA random position(s) is represented by at least one probe in each set of probes; and
wherein the base at the discriminating position is identical within each set of probes, but differs between the first and the second set of probes.

In the context of the present disclosure, the term “composition” may be understood in the broadest sense as any mixture that comprises a first set of probes and a second set of probes. The composition may further comprise a solvent suitable for the probes of the present disclosure. For example, the solvent may comprise water, an aqueous buffer (comprising, e.g., TAPS (3-{[tris(hydroxymethyl) methyl]amino} propanesulfonic acid), Bicine (N,N-bis(2-hydroxyethyl)glycine), Tris (tris(hydroxymethyl)methylamine), Tricine (tris(hydroxymethyl)methylglycine), HEPES (2-hydroxyethyl-1-piperazineethanesulfonic acid), TES (2-{[tris(hydroxymethyl)methyl]amino}ethanesulfonic acid, MOPS (3-(N-morpholino)propanesulfonic acid), PIPES (piperazine-N,N′-bis(2-ethanesulfonic acid)), Cacodylate (dimethylarsinic acid), SSC (saline sodium citrate), MES (2-(N-morpholino)ethanesulfonic acid), phosphate, hydrogen phosphate, dihydrogen phosphate, citrate, acetate, and/or borax), an organic solvent, (e.g. dimethyl sulphoxide (DMSO), dimethylformamide (DMF) or a combination thereof) or a combination thereof. Further, the composition may contain inorganic and/or organic salt(s), in particular the composition may contain magnesium, sodium, potassium, calcium, chloride, phosphate, hydrogen phosphate, dihydrogen phosphate, citrate, acetate, and/or borax salts. The composition may also comprise other substances, such as, e.g. biological substances (e.g., proteins, peptides, amino acids, saccharides, lipids, etc.), synthetic polymers (e.g., polyethylene imine (PEI), hydroxypropylmethacrylamide (HPMA), polyethylene glycol (PEG)), DNA-staining fluorescence dyes (e.g., SYBR green, ethidium bromide), one or more detergents, surfactants and/or emulgators (e.g., sodium dodecylsulfate (SDS)), one or more chelators (e.g., ethylenediaminetetraacetate (EDTA)), therapeutic agents, or combinations of two or more thereof. Alternatively, the composition may be dried or freeze-dried. Alternatively, the probes may also be immobilized on a solid support, such as an array surface and/or a bead surface.

As used in the context of the present disclosure, the term “nucleotide” may be understood in the broadest sense as a monomer of a deoxyribonucleic acid (DNA) or a locked nucleic acid (LNA) strand (or an LNA analogue). As will be understood by a person skilled in the art, each nucleotide of the probe comprises one nucleobase. The terms “base” and “nucleobase” as used herein may be understood interchangeably in the broadest sense as understood by the person skilled in the art. The nucleobase may be e.g., adenine (A), thymine (T), cytosine (C), guanine (G), uracil (U), methyl cytosine (mC) or an analogue thereof.

According to the instant disclosure, the nucleotides may be conjugated by ester formation of the 3′ and the 5′ hydroxyl groups of the ribose, deoxyribose and/or ribose derivatives (e.g., locked ribose) with phosphate anions as known in the art. According to some embodiments of the instant disclosure, the probes may comprise no other covalently bound molecular moieties linking the nucleotides of the strand with one another other than nucleotides and phosphate anion moieties. For example, according to such embodiments, there may be no linkers or spacers located between the nucleotides.

According to some embodiments of the present disclosure, the length of the plain nucleic acid strand may be eight nucleotides in length. It should be understood that this length refers to the length of the nucleic acid strand, but should not exclude that other molecular moieties (such as, e.g., one or more fluorophore(s), one or more quencher(s), one or more binding moiety/moieties or the like) may be added to the probe, in particular may be added at the end of the probe. The term “end” of the probe may be understood as the “3′ end” or the “5′ end” of the probe. Herein, the term “end” and the term “terminus” may be understood interchangeably. In particular, a molecular moiety may be conjugated to the 3′ and/or the 5′ hydroxyl group of the probe. The person skilled in the art will notice that the terms “5′ end” as used herein may refer to the 5′ end of the nucleotide strand, but may not exclude that at the 5′ end another molecular moiety (such as, e.g., a fluorophore, a quencher, a binding moiety or the like) is added to the 5′ end of the probe. The person skilled in the art will notice that the terms “3′ end” as used herein may refer to the 3′ end of the nucleotide strand, but may not exclude that at the 3′ end another molecular moiety (such as, e.g., a fluorophore, a quencher, a binding moiety or the like) is added to the 3′ end of the probe.

As used in the context of the present disclosure, the term “locked nucleic acid”(“LNA”), may be understood in the broadest sense as a nucleotide, wherein the ribose ring is “locked” with an extra bridge connecting the 2′-oxygen atom with the 4′-carbon atom of the nucleotide (e.g., a methylene bridge) (as described in WO 99/14226). Therefore, LNA may be understood as modified, inaccessible RNA, wherein the bridge “locks” the ribose in the 3′ endo confirmation. LNA nucleotides as well as LNA oligomers are commercially available. The locked ribose confirmation is known to enhance base stacking and backbone pre-organization, significantly increasing the hybridization properties with a DNA or RNA target strand and, therefore, increasing the binding strength per base pair (increased thermal stability/melting temperature). LNAs have been used for DNA microarrays, as FISH probes and as real-time PCR probes. LNAs are widely resistant to endo- and exonuclease activity. Alternative LNA nucleotides include ENA (2′-O,4′-C-ethylene-bridges nucleic acid) and 2′-amino-LNA derivatives (described in K. Morita et al., Bioorg. Med. Chem. Lett. 2002, 12, 73-76 and S. K. Singh et al., J. Org. Chem. 1998, 63, 10035) and 5′ Methyl LNA derivatives (described in WO 2010/077578). Further alternatives are Blocked Nucleic Acids BNA (described in U.S. Pat. Nos. 7,427,672 and 7,217,805) and bicyclic cyclohexitol nucleic acid (described in WO 2009/100320). LNAs may also be combined with other nucleotides, such as DNA nucleotides. Such oligomers are commercially available. As used herein, the nucleic acid strand as used in the context of the present disclosure is a molecule comprising “n” number of DNA nucleotides (e.g., n=1, 2, or 3, etc.) and 8 minus n locked nucleic acid (LNA) nucleotides.

As used herein, the terms “first set of probes” and “second set of probes” refer to two sets of probes, wherein both sets of probes have identical nucleotide sequences except for the base(s) at the LNA random position(s); and the base at a discriminating position.

In the context of the present disclosure, the term “LNA random position” refers to a position in the nucleotide sequence, wherein the nucleobase is any nucleotide of the group consisting of adenine (A), thymine (T), cytosine (C) or guanine (G). Alternatively, the nucleobase may also be uracil (U) or methyl cytosine (mC) or another nucleobase that can form a base pair with a complementary nucleobase. In the context of the present disclosure, the terms “random position” and “wobble position” may be understood interchangeably. The nucleobase is independently selected from the nucleobases A, T, C and G (it is also possible the complementary nucleobase is U). In the context of the present disclosure, there may be one, two, or three LNA random position(s) in each probe of the first and the second set of probes. It will be understood that a set of probes according to the present disclosure may contain 4ⁿ probes of different sequences, wherein n refers to the number of LNA random position(s). Thus, when there is one random position, as exemplified below, n=1 and 4¹=4, the set of probes would contain 4 different probes. Accordingly, when there are two LNA random positions in each probe, n=2 and 4²=16, the set of probes would contain 16 different probes and when there are three LNA random positions in each probe, n=3 and 4³=64, the set of probes would contain 64 different probes. The one, two, or three LNA random position(s) are located at the identical position in all probes of the first and the second set.

According to an exemplified embodiment of the instant disclosure, probe design of a composition comprising a first set of probes and a second set of probes having one LNA random position may, for example, may have the following sequences (according to this exemplified example, the first set of probes may comprise a mixture of the following probes):

5′-¹C_{(DNA)}-²G_{(LNA)}-³T_{(LNA)}-⁴A_{(LNA)}-⁵A_{(LNA)}-⁶G_{(LNA)}-⁷T_{(LNA)}-⁸A_{(LNA)}-3′,

5′-¹C_{(DNA)}-²G_{(LNA)}-³T_{(LNA)}-⁴A_{(LNA)}-⁵A_{(LNA)}-⁶G_{(LNA)}-⁷T_{(LNA)}-⁸T_{(LNA)}-3′,

5′-¹C_{(DNA)}-²G_{(LNA)}-³T_{(LNA)}-⁴A_{(LNA)}-⁵A_{(LNA)}-⁶G_{(LNA)}-⁷T_{(LNA)}-⁸C_{(LNA)}-3′,

5′-¹C_{(DNA)}-²G_{(LNA)}-³T_{(LNA)}-⁴A_{(LNA)}-⁵A_{(LNA)}-⁶G_{(LNA)}-⁷T_{(LNA)}-⁸G_{(LNA)}-3′,

According to this exemplified example, the corresponding second set of probes may comprise a mixture of the following probes:

5′-¹C_{(DNA)}-²G_{(LNA)}-³T_{(LNA)}-⁴A_{(LNA)}-⁵A_{(LNA)}-⁶G_{(LNA)}-⁷T_{(LNA)}-⁸A_{(LNA)}-3′,

5′-¹C_{(DNA)}-²G_{(LNA)}-³T_{(LNA)}-⁴A_{(LNA)}-⁵A_{(LNA)}-⁶G_{(LNA)}-⁷T_{(LNA)}-⁸T_{(LNA)}-3′,

5′-¹C_{(DNA)}-²G_{(LNA)}-³T_{(LNA)}-⁴A_{(LNA)}-⁵A_{(LNA)}-⁶G_{(LNA)}-⁷T_{(LNA)}-⁸C_{(LNA)}-3′,

5′-¹C_{(DNA)}-²G_{(LNA)}-³T_{(LNA)}-⁴A_{(LNA)}-⁵A_{(LNA)}-⁶G_{(LNA)}-⁷T_{(LNA)}-⁸G_{(LNA)}-3′,

With regard to the exemplified sets of probes, the superscript digits 1-8 characterize the nucleotide position in the probe from the 5′ end. The subscript three-letter codes in parentheses indicate whether the nucleotide is a deoxyribonucleic acid (DNA) nucleotide or a locked nucleic acid (LNA) nucleotide.

As can be seen from the above example, the probes are eight nucleotides in length. The nucleotide in position 1 from the 5′ end is a DNA nucleotide. The nucleotides in positions 2-8 from the 5′ end are LNA nucleotides. The LNA random position is located in position 8 from the 5′ end. Therefore, each set of probes contains four different probes. In the above example, the discriminating position is located at position 4 from the 5′ end of the probes. All four probes of the first set of probes bear an adenine (A) nucleobase at the discriminating position (position 4 from the 5′ end), whereas all four probes of the second set of probes bear a cytosine (C) nucleobase at the discriminating position.

It will be understood that the above example is intended to explain the probe design of a typical probe of the present disclosure, but is not intended to limit the scope of the present disclosure.

As used herein, the term “discriminating position” refers to a position in the probe, where a nucleotide differs between the sets of probes. Stated another way, the nucleobase at the discriminating position within a set of probes is identical, but differs from the nucleobase at the discriminating position in the other sets of probes. It should be understood that an adenine moiety may be replaced by thymine, cytosine or guanine; a thymine moiety may be replaced by guanine, cytosine or adenine; a cytosine moiety may be replaced by thymine, guanine or adenine; and a guanine moiety may be replaced by thymine, cytosine or adenine. In the context of the present disclosure, the discriminating position is located at the identical position in all probes of the probe set.

The term “identical position,” as used herein and unless otherwise indicated herein, refers to a position in the probe sequence. In the context of the present disclosure, the term “position” refers to a nucleotide position from the 5′ end as regularly used for nucleic acid sequences. The term “identical sequence” refers to two sequences that each have the same type of nucleotide, thus, also the same type of nucleobase, at an identical position of the probe.

According to embodiments of the instant disclosure, the composition may comprise two, three, four, five or more different sets of probes that may each detect a particular genotype of a particular locus. In the context of the present disclosure, the term “locus” may be understood in the broadest sense as a position in the nucleotide sequence of a gene, in particular a target gene to which the probe of the disclosure may bind. The locus may be also designated as the part of the target sequence in question or the target sequence. A potential mutation may be located at the locus. Alternatively, the locus does not comprise a mutation. The locus may be a gene wherein a frameshift mutation occurs, a part of a gene wherein a frameshift mutation occurs, a group of two, three, four, five or more nucleotides or a single nucleotide.

The term “genotype” may be understood in the broadest sense as the genetic makeup of an organism or a virus (i.e. the specific allele makeup of the organism or virus). As used herein, the term “organism” refers to all living beings, such as bacteria, archaebacteria, animals, plants, fungi. Further the term organism may refer to the dead body of a being that has been a living being before. The term “virus” may include virus-like particles.

The term “mutation” as used herein, may be understood in the broadest sense as an alteration in the nucleotide sequence. A mutation may occur in an encoding section of the genome or may occur in a non-encoding section of the genome. A mutation occurring in an encoding section of the genome may result in an altered amino acid sequence of a polypeptide encoded by said gene when the gene is expressed. Alternatively, a mutation may be a silent mutation, wherein the amino acid sequence of the expressed polypeptide is not affected or wherein the mutation occurs in a non-encoding region of the genome. A mutation may occur naturally in a population. Alternatively, a mutation may be provoked by xenobiotics or radiation. Such xenobiotics may be a mutagenic agent as known in the art (e.g., alkylating agent (e.g., nitrogen mustards (e.g., Cyclophosphamide, Mechlorethamine or mustine (HN2), Uramustine or uracil mustard, Melphalan, Chlorambucil, Ifosfamide), nitrosoureas (e.g., Carmustine, Lomustine, Streptozocin), alkyl sulfonates (e.g., Busulfan), thiotepa and its analogues, platinum derivates (e.g., Cisplatin, Carboplatin, Nedaplatin, Oxaliplatin, Satraplatin, Triplatin tetranitrate), Procarbazine, altretamine, aflatoxine and aflatoxine and metabolic products and derivatives thereof, nitrite, aniline and metabolic products and derivatives thereof, benzene and metabolic products and derivatives thereof, polycyclic aromatics and metabolic products and derivatives thereof) to a nucleobase), nitrosamines, arsenic, asbestos, beryllium and its compounds, ethylene oxide, hexavalent chromium(VI) compounds, radon, vinyl chloride, smoking, etc.). Such radiation may be, for example, ultra violet (UV) radiation, X-ray radiation, radioactive/nuclear radiation (e.g., alpha-, beta- or gamma-radiation) or cosmic radiation.

Herein, the term “gene” may be understood in the broadest sense, as known in the art, as a unit of the nucleotide sequence of a genome as known in the art. The genome is the entity of the genes of an organism.

A mutation may be a frameshift mutation, a deletion of a gene or a part of the gene, the repetition of a gene, the insertion of a gene, or a single nucleotide exchange. Additionally, the conjugation of molecular moieties to one or more nucleobases may also be understood as a mutation, in particular when said conjugation may lead to alterations in the transcription and/or translation product. Such conjugation may be any conjugation known in the art such as methylation of a nucleobase, loss of a methylene group of a nucleobase, conjugation of an alkylating agent (e.g., nitrogen mustards (e.g., Cyclophosphamide, Mechlorethamine or mustine (HN2), Uramustine or uracil mustard, Melphalan, Chlorambucil, Ifosfamide), nitrosoureas (e.g., Carmustine, Lomustine, Streptozocin), alkyl sulfonates (e.g., Busulfan), thiotepa and its analogues, platinum derivates (e.g., Cisplatin, Carboplatin, Nedaplatin, Oxaliplatin, Satraplatin, Triplatin tetranitrate), Procarbazine, altretamine, aflatoxine and aflatoxine and metabolic products and derivatives thereof, nitrite, nitrosamine, aniline and metabolic products and derivatives thereof, benzene and metabolic products and derivatives thereof, polycyclic aromatics and metabolic products and derivatives thereof) to a nucleobase.

According to an embodiment of the present disclosure, the mutation comprises a deletion of a gene or a part of the gene, the repetition of a gene, or a single nucleotide exchange. In some embodiments, such mutation is a single nucleotide exchange (SNP).

A mutation may result in an altered nucleotide sequence, such as an altered DNA sequence. Therefore, a mutation may result in different alleles of a gene. Alternatively, a mutation may result in different alleles in a non-encoding part of the genome. The existence of at least two different alleles may be understood as a polymorphism. However, a polymorphism may also occur naturally throughout the population. Therefore, the term “polymorphism” as used herein may be understood in the broadest sense as the occurrence of different genotypes of a specific gene. The term “genotype” may be understood in the broadest sense as the nucleotide sequences in a gene. The different forms of a gene occurring due to a polymorphism, thus, the polymorphic forms may also be designated as “alleles”. Throughout the population, there may be two, three, four, five, six or more different alleles of a gene. As used in the context of the present disclosure, the term “polymorphism” is not used to refer to certain incidence of a mutated gene throughout the population and may be used to refer to a single individual that shows a specific genotype.

The polymorphism may result in different phenotypes or may be silent. Herein, the term “silent” means that, though there are different genotypes, the phenotypes are not affected or are at least not distinguishable by the methods known in the art.

According to embodiments of the present disclosure, the polymorphism leads to different phenotypes. Some phenotypes may lead to certain diseases or pathologic conditions that occur as a direct result of the altered nucleotide sequences (e.g., cancer, sickle-cell anaemia, hypercoagulability). Alternatively, different phenotypes may result in differences in the predisposition to certain diseases or pathologic conditions (e.g., cancer, autism, schizophrenia, diabetes mellitus) and/or different phenotypes may result in differences in the metabolism, such as xenobiotic metabolizing enzyme polymorphisms in the alcohol dehydrogenase, aldehyde dehydrogenase, glutathione-S-transferase, glucoronosyl transferase or a member of the cytochrome P 450 (CYP) superfamily.

A polymorphism that results from a single nucleotide exchange may be designated as a single nucleotide polymorphism (SNP). A polymorphism that results from a deletion of a gene or a part thereof or the alteration in the copy numbers of a gene may be designated as a copy number variant (CNV).

According to the instant disclosure single discriminating position (of the probes comprising the probe sets) may be used to detect a point mutation on a certain locus of the target DNA. As used herein the terms “point mutation,” “single base pair mutation,” “single base substitution,” or other expressions known by those skilled in the art may be understood interchangeably. The position on the target gene may be understood as the locus of interest.

In an embodiment of the present disclosure, the discriminating position is at position 2, 3, 4, 5, 6 or 7 from the 5′ end in each probe. In some embodiments. The discriminating position is at position 3, 4, or 5. In some embodiments, the discriminating position is at position 4.

As disclosed herein, according to the instant disclosure, the nucleotide at position 1 from the 5′ end may be an LNA nucleotide or a DNA nucleotide. According to exemplified embodiments disclosed herein the nucleotide at position 1 from the 5′ end comprises a DNA nucleotide.

Each probe of the present disclosure may comprise from one to three DNA nucleotides and from five to seven LNAs. Additionally, the probes disclosed herein may further comprise one or more non-nucleotide moiety/moieties such as one or more fluorophore(s), one or more quencher(s), one or more linker(s) (e.g., an alkyl linker, a PEG linker, a peptidic linker, a saccharide linker or the like), one or more non-fluorescent dye(s) (e.g., a dinitrophenyl moiety, Malachite Green or the like), one or more binding moiety/moieties that can bind to other molecules (e.g., a maleimide, an isothiocyanate, an active ester (e.g., succinimidyl ester, p-nitrophenylester) or the like), and/or one or more moiety/moieties selectively binding to high-molecular weight molecules (e.g., biotin, methotrexate, glycocorticoids or the like). According to embodiments of the instant disclosure, a probe conjugated with biotin, for example, may be detected by using labeled strepavidine. A probe (according to the instant disclosure) conjugated to methotrexate, for example, may be detected by using labeled dihydrofolate reductase (DHFR). In other embodiments, a probe conjugated with a glycocorticoid, for example, may be detected by an antibody or an antibody derivative (e.g., Fab fragment, a single chain antibody, a diabody, a triabody, a tandab or the like).

According to some embodiments of the present disclosure, each probe (of the sets of probes) may consist of:

one DNA and seven LNA nucleotides,

two DNA and six LNA nucleotides, or

three DNA and five LNA nucleotides.

It should be understood that as used herein, the term “consists of” merely refers to the nucleotide content of the probes, but does not exclude that the probe may further comprise non-nucleotide moieties such as, fluorophores, quenchers, binding molecules, linkers and the like, as disclosed herein.

According to some embodiments of the present disclosure, the one, two or three DNA nucleotides of the probe may be located near the 5′ end of said probe. In some such embodiments, at least one of the five 5′-terminal nucleotides is a DNA nucleotide. In various embodiments (separately described below) of such embodiments of the present disclosure:

the probe has a DNA nucleotide in position 1 from the 5′ end,

the probe has a DNA nucleotide in position 2 from the 5′ end,

the probe has a DNA nucleotide in position 3 from the 5′ end,

the probe has a DNA nucleotide in position 4 from the 5′ end,

the probe has a DNA nucleotide in position 5 from the 5′ end,

the probe has DNA nucleotides in positions 1 and 2 from the 5′ end,

the probe has DNA nucleotides in positions 1 and 3 from the 5′ end,

the probe has DNA nucleotides in positions 1 and 4 from the 5′ end,

the probe has DNA nucleotides in positions 1 and 5 from the 5′ end,

the probe has DNA nucleotides in positions 2 and 3 from the 5′ end,

the probe has DNA nucleotides in positions 2 and 4 from the 5′ end,

the probe has DNA nucleotides in positions 2 and 5 from the 5′ end,

the probe has DNA nucleotides in positions 3 and 4 from the 5′ end,

the probe has DNA nucleotides in positions 3 and 5 from the 5′ end,

the probe has DNA nucleotides in positions 4 and 5 from the 5′ end,

the probe has DNA nucleotides in positions 1, 2 and 3 from the 5′ end,

the probe has DNA nucleotides in positions 1, 2 and 4 from the 5′ end,

the probe has DNA nucleotides in positions 1, 2 and 5 from the 5′ end,

the probe has DNA nucleotides in positions 1, 3 and 4 from the 5′ end,

the probe has DNA nucleotides in positions 1, 3 and 5 from the 5′ end,

the probe has DNA nucleotides in positions 1, 4 and 5 from the 5′ end,

the probe has DNA nucleotides in positions 2, 3 and 4 from the 5′ end,

the probe has DNA nucleotides in positions 2, 4 and 5 from the 5′ end,

the probe has DNA nucleotides in positions 3, 4 and 5 from the 5′ end,

the probe has DNA nucleotides in positions 1, 2, 3 and 4 from the 5′ end,

the probe has DNA nucleotides in positions 2, 3, 4 and 5 from the 5′ end,

the probe has DNA nucleotides in positions 1, 3, 4 and 5 from the 5′ end,

the probe has DNA nucleotides in positions 1, 2, 4 and 5 from the 5′ end,

the probe has DNA nucleotides in positions 1, 2, 3 and 5 from the 5′ end, or

the probe has DNA nucleotides in positions 1, 2, 3, 4 and 5 from the 5′ end.

In some such embodiments, at least one of the four 5′-terminal nucleotides is a DNA nucleotide. In various embodiments (separately described below) of such embodiments of the present disclosure:

the probe has a DNA nucleotide in position 1 from the 5′ end,

the probe has a DNA nucleotide in position 2 from the 5′ end,

the probe has a DNA nucleotide in position 3 from the 5′ end,

the probe has a DNA nucleotide in position 4 from the 5′ end,

the probe has DNA nucleotides in positions 1 and 2 from the 5′ end,

the probe has DNA nucleotides in positions 1 and 3 from the 5′ end,

the probe has DNA nucleotides in positions 1 and 4 from the 5′ end,

the probe has DNA nucleotides in positions 2 and 3 from the 5′ end,

the probe has DNA nucleotides in positions 2 and 4 from the 5′ end,

the probe has DNA nucleotides in positions 3 and 4 from the 5′ end,

the probe has DNA nucleotides in positions 1, 2 and 3 from the 5′ end,

the probe has DNA nucleotides in positions 1, 2 and 4 from the 5′ end,

the probe has DNA nucleotides in positions 1, 3 and 4 from the 5′ end,

the probe has DNA nucleotides in positions 2, 3 and 4 from the 5′ end, or

the probe has DNA nucleotides in positions 1, 2, 3 and 4 from the 5′ end.

In some further embodiments, at least one of the three 5′-terminal nucleotides is a DNA nucleotide. In various embodiments (separately described below) of such embodiments of the present disclosure:

the probe has a DNA nucleotide in position 1 from the 5′ end,

the probe has a DNA nucleotide in position 2 from the 5′ end,

the probe has a DNA nucleotide in position 3 from the 5′ end,

the probe has DNA nucleotides in positions 1 and 2 from the 5′ end,

the probe has DNA nucleotides in positions 1 and 3 from the 5′ end,

the probe has DNA nucleotides in positions 2 and 3 from the 5′ end, or

the probe has DNA nucleotides in positions 1, 2 and from the 5′ end.

In even further embodiments of the instant disclosure, at least one of the two 5′-terminal nucleotides is a DNA nucleotide. In various embodiments (separately described below) of such embodiments of the present disclosure:

the probe has a DNA nucleotide in position 1 from the 5′ end,

the probe has a DNA nucleotide in position 2 from the 5′ end, or

the probe has DNA nucleotides in positions 1 and 2 from the 5′ end.

According to an exemplary embodiment of the instant disclosure, only the nucleotide at position 1 from the 5′ end is a DNA nucleotide.

According to some embodiments of the instant disclosure, the 5 to 7 nucleotides at positions 1, 2, 3, 4, 5, 6, 7 and/or 8 from the 5′ end may be LNA nucleotides. In some such embodiments, at least the nucleotides at positions 4, 5 and 6 from the 5′ end are LNA nucleotides, for example, in some embodiments the nucleotides at positions 4, 5, 6 and 7 from the 5′ end are LNA nucleotides, and if even other embodiments at least the nucleotides at positions 4, 5, 6, 7 and 8 from the 5′ end are LNA nucleotides. In yet further embodiments, at least the nucleotides at positions 3, 4, 5, 6, 7 and 8 from the 5′ end are LNA nucleotides, and even further embodiments the nucleotides at positions 2, 3, 4, 5, 6, 7 and 8 from the 5′ end are LNA nucleotides.

According to an exemplary embodiment of the instant disclosure, the nucleotide at position 1 from the 5′ end is a DNA nucleotide and the nucleotides in positions 2, 3, 4, 5, 6, 7, and 8 from the 5′ end are LNA nucleotides.

According to the instant disclosure, in each position, the probe may feature a determined nucleotide or a random nucleotide. The positions may be the same for all probes of a set of probes. As disclosed herein, the composition comprises at least two set of probes, as defined herein, and may comprise one, two, three, four or more set of probes.

As used herein, the term “determined nucleotide” refers to a position of a certain nucleotide in the probe, wherein the type of the nucleotide (e.g., adenine (A), thymine (T), cytosine (C), guanine (G), uracil (U), 5-methylcytosine (mC)) is known. According to the instant disclosure, the nucleobase at a determined position may be A, T, C, G or U.

According to some embodiments of the instant disclosure, the nucleotides at the 5′ end are determined. In various embodiments (separately described below) of such embodiments of the present disclosure:

at least nucleotide at position 1 from the 5′ end is determined,

at least nucleotide at position 2 from the 5′ end is determined,

at least nucleotide at position 3 from the 5′ end is determined,

at least nucleotide at position 4 from the 5′ end is determined,

at least nucleotide at position 5 from the 5′ end is determined,

at least nucleotide at position 6 from the 5′ end is determined,

at least nucleotides at position 1 and 2 from the 5′ end are determined,

at least nucleotides at position 1 and 3 from the 5′ end are determined,

at least nucleotides at position 1 and 4 from the 5′ end are determined,

at least nucleotides at position 1 and 5 from the 5′ end are determined,

at least nucleotides at position 1 and 6 from the 5′ end are determined,

at least nucleotides at position 2 and 3 from the 5′ end are determined,

at least nucleotides at position 2 and 4 from the 5′ end are determined,

at least nucleotides at position 2 and 5 from the 5′ end are determined,

at least nucleotides at position 2 and 6 from the 5′ end are determined,

at least nucleotides at position 3 and 4 from the 5′ end are determined,

at least nucleotides at position 3 and 5 from the 5′ end are determined,

at least nucleotides at position 3 and 6 from the 5′ end are determined,

at least nucleotides at position 4 and 5 from the 5′ end are determined,

at least nucleotides at position 4 and 6 from the 5′ end are determined,

at least nucleotides at position 5 and 6 from the 5′ end are determined,

at least nucleotides at position 1, 2 and 3 from the 5′ end are determined,

at least nucleotides at position 1, 2 and 4 from the 5′ end are determined,

at least nucleotides at position 1, 2 and 5 from the 5′ end are determined,

at least nucleotides at position 1, 2 and 6 from the 5′ end are determined,

at least nucleotides at position 1, 3 and 4 from the 5′ end are determined,

at least nucleotides at position 1, 3 and 5 from the 5′ end are determined,

at least nucleotides at position 1, 3 and 6 from the 5′ end are determined,

at least nucleotides at position 1, 4 and 5 from the 5′ end are determined,

at least nucleotides at position 1, 4 and 6 from the 5′ end are determined,

at least nucleotides at position 1, 5 and 6 from the 5′ end are determined,

at least nucleotides at position 2, 3 and 4 from the 5′ end are determined,

at least nucleotides at position 2, 3 and 5 from the 5′ end are determined,

at least nucleotides at position 2, 3 and 6 from the 5′ end are determined,

at least nucleotides at position 2, 4 and 5 from the 5′ end are determined,

at least nucleotides at position 2, 4 and 6 from the 5′ end are determined,

at least nucleotides at position 2, 5 and 6 from the 5′ end are determined,

at least nucleotides at position 3, 4 and 5 from the 5′ end are determined,

at least nucleotides at position 3, 4 and 6 from the 5′ end are determined,

at least nucleotides at position 3, 5 and 6 from the 5′ end are determined,

at least nucleotides at position 4, 5 and 6 from the 5′ end are determined,

at least nucleotides at position 1, 2, 3 and 4 from the 5′ end are determined,

at least nucleotides at position 1, 2, 3 and 5 from the 5′ end are determined,

at least nucleotides at position 1, 2, 3 and 6 from the 5′ end are determined,

at least nucleotides at position 1, 2, 4 and 5 from the 5′ end are determined,

at least nucleotides at position 1, 2, 4 and 6 from the 5′ end are determined,

at least nucleotides at position 1, 2, 5 and 6 from the 5′ end are determined,

at least nucleotides at position 1, 3, 4 and 5 from the 5′ end are determined,

at least nucleotides at position 1, 3, 4 and 6 from the 5′ end are determined,

at least nucleotides at position 2, 3, 4 and 5 from the 5′ end are determined,

at least nucleotides at position 2, 3, 4 and 6 from the 5′ end are determined,

at least nucleotides at position 2, 3, 5 and 6 from the 5′ end are determined,

at least nucleotides at position 1, 2, 3, 4 and 5 from the 5′ end are determined,

at least nucleotides at position 1, 2, 3, 4 and 6 from the 5′ end are determined,

at least nucleotides at position 1, 2, 3, 5 and 6 from the 5′ end are determined,

at least nucleotides at position 1, 2, 4, 5 and 6 from the 5′ end are determined,

at least nucleotides at position 1, 3, 4, 5 and 6 from the 5′ end are determined,

at least nucleotides at position 2, 3, 4, 5 and 6 from the 5′ end are determined, or

at least nucleotides at position 1, 2, 3, 4, 5 and 6 from the 5′ end are determined.

As disclosed herein, embodiments of the instant disclosure include at least the nucleotide at position 1 from the 5′ end being determined., Also, in various embodiments, at least the nucleotides at positions 1 and 2 from the 5′ end may be determined, at least the nucleotides at positions 1, 2 and 3 from the 5′ end may be determined, at least the nucleotides at positions 1, 2, 3 and 4 from the 5′ end may be determined, at least the nucleotides at positions 1, 2, 3, 4 and 5 or the nucleotides at positions 1, 2, 3, 4, 5 and 6 from the 5′ end may also be determined.

Further, according to the instant disclosure, the random position(s) may be located at position(s) 5, 6, 7, or 8 from the 5′ end. According to some embodiments, two random positions may be located at positions 5 and 6; 5 and 7; 5 and 8; 6 and 7; 6 and 8; or 7 and 8 from the 5′ end. According to the instant disclosure, three random positions may be located at positions 5, 6, and 7 from the 5′ end; positions 5, 6, and 8 from the 5′ end; positions 5, 7, and 8 from the 5′ end; positions 6, 7, and 8 from the 5′ end. Further, according to embodiments of the instant disclosure, four random positions may be located at positions 5, 6, 7 and 8 from the 5′ end. In some exemplary embodiments provided herein, two random positions are located in positions 7 and 8 from the 5′ end and three random positions are located in positions 6, 7 and 8 from the 5′ end.

In an embodiment of the instant disclosure, the composition of the present disclosure is characterized in that,
a) the set of probes has one LNA random position located at position 5; 6; 7; or 8 from the 5′ end; or
b) the set of probes has two LNA random positions located at positions 5 and 6; 5 and 7; 5 and 8; 6 and 7; 6 and 8; or 7 and 8 from the 5′ end, preferably at positions 7 and 8; or
c) the set of probes has three LNA random positions located at positions 5, 6 and 7; 5, 6 and 8; or 6, 7 and 8 from the 5′ end, preferably at positions 6, 7 and 8.

Exemplary probes according to the instant disclosure may have the general structure of:

5′-D-L-L-L-L-L-L-X-3′,
5′-D-L-L-L-L-L-X-L-3′,
5′-D-L-L-L-L-X-L-L-3′,
5′-D-L-L-L-X-L-L-L-3′,
5′-D-L-L-L-L-L-X-X-3′,
5′-D-L-L-L-L-X-L-X-3′,
5′-D-L-L-L-X-L-L-X-3′,
5′-D-L-L-L-L-X-X-L-3′,
5′-D-L-L-L-X-L-X-L-3′,
5′-D-L-L-L-X-X-L-L-3′,
5′-D-L-L-L-L-X-X-X-3′,
5′-D-L-L-L-X-L-X-X-3′,
5′-D-L-L-L-X-X-L-X-3′,
5′-D-L-L-L-X-X-X-L-3′,
5′-L-D-L-L-L-L-L-X-3′,
5′-L-D-L-L-L-L-X-L-3′,
5′-L-D-L-L-L-X-L-L-3′,
5′-L-D-L-L-X-L-L-L-3′,
5′-L-D-L-L-L-L-X-X-3′,
5′-L-D-L-L-L-X-L-X-3′,
5′-L-D-L-L-X-L-L-X-3′,
5′-L-D-L-L-L-X-X-L-3′,
5′-L-D-L-L-X-L-X-L-3′,
5′-L-D-L-L-X-X-L-L-3′,
5′-L-D-L-L-L-X-X-X-3′,
5′-L-D-L-L-X-L-X-X-3′,
5′-L-D-L-L-X-X-L-X-3′,
5′-L-D-L-L-X-X-X-L-3′,
5′-L-L-D-L-L-L-L-X-3′,
5′-L-L-D-L-L-L-X-L-3′,
5′-L-L-D-L-L-X-L-L-3′,
5′-L-L-D-L-X-L-L-L-3′,
5′-L-L-D-L-L-L-X-X-3′,
5′-L-L-D-L-L-X-L-X-3′,
5′-L-L-D-L-X-L-L-X-3′,
5′-L-L-D-L-L-X-X-L-3′,
5′-L-L-D-L-X-L-X-L-3′,
5′-L-L-D-L-X-X-L-L-3′,
5′-L-L-D-L-L-X-X-X-3′,
5′-L-L-D-L-X-L-X-X-3′,
5′-L-L-D-L-X-X-L-X-3′,
5′-L-L-D-L-X-X-X-L-3′,
5′-L-L-L-D-L-L-L-X-3′,
5′-L-L-L-D-L-L-X-L-3′,
5′-L-L-L-D-L-X-L-L-3′,
5′-L-L-L-D-X-L-L-L-3′,
5′-L-L-L-D-L-L-X-X-3′,
5′-L-L-L-D-L-X-L-X-3′,
5′-L-L-L-D-X-L-L-X-3′,
5′-L-L-L-D-L-X-X-L-3′,
5′-L-L-L-D-X-L-X-L-3′,
5′-L-L-L-D-X-X-L-L-3′,
5′-L-L-L-D-L-X-X-X-3′,
5′-L-L-L-D-X-L-X-X-3′,
5′-L-L-L-D-X-X-L-X-3′,
5′-L-L-L-D-X-X-X-L-3′,
5′-L-L-L-L-D-L-L-X-3′,
5′-L-L-L-L-D-L-X-L-3′,
5′-L-L-L-L-D-X-L-L-3′,
5′-L-L-L-L-X-D-L-L-3′,
5′-L-L-L-L-D-L-X-X-3′,
5′-L-L-L-L-D-X-L-X-3′,
5′-L-L-L-L-X-D-L-X-3′,
5′-L-L-L-L-D-X-X-L-3′,
5′-L-L-L-L-X-D-X-L-3′,
5′-L-L-L-L-X-X-D-L-3′,
5′-L-L-L-L-D-X-X-X-3′,
5′-L-L-L-L-X-D-X-X-3′,
5′-L-L-L-L-X-X-D-X-3′,
5′-L-L-L-L-X-X-X-D-3′,
5′-L-L-L-L-L-D-L-X-3′,
5′-L-L-L-L-L-D-X-L-3′,
5′-L-L-L-L-L-X-D-L-3′,
5′-L-L-L-L-X-L-D-L-3′,
5′-L-L-L-L-L-D-X-X-3′,
5′-L-L-L-L-L-X-D-X-3′,
5′-L-L-L-L-X-L-D-X-3′,
5′-L-L-L-L-L-X-X-D-3′,
5′-L-L-L-L-X-D-X-L-3′,
5′-L-L-L-L-X-X-L-D-3′,
5′-L-L-L-L-L-L-D-X-3′,
5′-L-L-L-L-L-L-X-D-3′,
5′-L-L-L-L-L-X-L-D-3′,
5′-L-L-L-L-X-L-L-D-3′,
5′-L-L-L-L-L-D-X-X-3′,
or
5′-L-L-L-L-X-L-X-D-3′,

wherein D comprises a DNA nucleotide, L comprises a LNA nucleotide, and X comprises a LNA random position.

According to some embodiments, the probe may have the general structure:

5′-D-L-L-L-L-L-L-X-3′,
5′-D-L-L-L-L-L-X-L-3′,
5′-D-L-L-L-L-X-L-L-3′,
5′-D-L-L-L-X-L-L-L-3′,
5′-D-L-L-L-L-L-X-X-3′,
5′-D-L-L-L-L-X-L-X-3′,
5′-D-L-L-L-X-L-L-X-3′,
5′-D-L-L-L-L-X-X-L-3′,
5′-D-L-L-L-X-L-X-L-3′,
5′-D-L-L-L-X-X-L-L-3′,
5′-D-L-L-L-L-X-X-X-3′,
5′-D-L-L-L-X-L-X-X-3′,
5′-D-L-L-L-X-X-L-X-3′,
or
5′-D-L-L-L-X-X-X-L-3′,

wherein D comprises a DNA nucleotide, L comprises a LNA nucleotide, and X comprises a LNA random position.

According to some embodiments, the probe may have the general structure:

5′-D-L-L-L-L-L-L-X-3′,
5′-D-L-L-L-L-L-X-X-3′,
or
5′-D-L-L-L-L-X-X-X-3′,

wherein D comprises a DNA nucleotide, L comprises a LNA nucleotide, and X comprises a LNA random position.

According to an embodiment of the present disclosure, the probes have the general structure 5′-D-L-L-L-L-X-X-X-3′, wherein D is a DNA nucleotide, each L is a LNA nucleotide and each X is a LNA random position.

In another embodiment of the present disclosure, the probes have the general structure 5′-D L LLL L X X-3′, wherein D is a DNA nucleotide, each L is a LNA nucleotide and each X is a LNA random position.

As disclosed herein, the probes may be labeled. According to some embodiments, the probes of different sets of probes are labeled differently.

In some embodiments of the present disclosure, the probes of the first set of probes are labeled with a first marker and the probes of the second set of probes are labeled with a second marker, wherein the first marker is different from the second marker.

In the context of the present disclosure, the term “marker” may be understood interchangeably with “label” or “detectable moiety” as any molecule or moiety that enable the discrimination of the probe from other molecules by any means known in the art. As used herein, the first and/or the second marker may be selected from the group consisting of, but not limited to fluorescent markers, quenchers, non-fluorescent dyes, binding moieties, radioactive atoms (e.g., ³H, ³²P, ³⁵S, ¹⁴C or lanthonoids), or heavy atoms (e.g., ²H or ¹³C).

According to some embodiments, the first and the second markers are fluorescent markers.

As used herein, the terms “fluorescent marker”, “fluorescence marker”, “fluorescent label”, “fluorescence label”, “fluorescent dye”, “fluorescence dye”, “fluorophore” and “fluorescent moiety” may be understood interchangeably. A fluorescent marker may be understood in the broadest sense as a molecular moiety that emits light when it is excited by light of another wavelength. Typically the wavelength that is emitted by the fluorophore is shifted to longer wavelength in comparison to the excitation light. This shift is known as “Stokes-Shift” or “Stokes shift” to those skilled in the art. The Stokes shift can be less than 5 nm, more than 5 nm, more than 10 nm, more than 20 nm, more than 30 nm, more than 50 nm, more than 75 nm, more than 100 nm, more than 150 nm, more than 200 nm, more than 250 nm, more than 300 nm, or even more than 400 nm. The one or more absorbance maxima of the fluorophore suitable for fluorescent detection may at a wavelength from 100-280 nm (UV-C light), 280-315 nm (UV-B light), 315 nm-400 nm (UV-A light), 400-750 nm (visible light), 750-1400 nm (IR-A light), 1400-3000 nm (IR-B-light). The one or more excitation maxima of the fluorophore suitable for fluorescent detection may 100-280 nm (UV-C light), 280-315 nm (UV-B light), 315 nm-400 nm (UV-A light), 400-750 nm (visible light), 750-1400 nm (IR-A light), 1400-3000 nm (IR-B-light). According to some embodiments, the absorbance and excitation maxima of the fluorescence are between 400 and 750 nm.

Fluorescent markers may be, e.g., fluorescein, fluorescein isothiocyanate (FITC), carboxyfluorescein, fluorescein derivatives, rhodamine dyes (Rhodamine, Rhodamine B, Rhodamine 6G, tetramethylrhodamine (TAMRA), rhodamine isothiocyanate and other Rhodamine derivatives) cyanine dyes (e.g., Cy3, Cy3.5, Cy5, Cy5.5, Cy7) and derivatives thereof, LC dyes (e.g., LC-Yellow 555, LC-Red 610, LC-Red 640, LC-Red 670, LC-Red 705) and derivatives thereof, Alexa dyes (Alexa 488, Alexa 546, Alexa 647) and derivatives thereof, S0387, HOECHST dye and derivatives thereof, erythrosine isothiocyanate and derivatives thereof, Oregon Green and derivatives thereof, Lucifer Yellow and derivatives thereof, phycoerythrin and derivatives thereof, FAM and derivatives thereof, LightCycler® Yellow 555 and derivatives thereof, VIC and derivatives thereof, HEX and derivatives thereof or quantum dots and derivatives thereof.

In the context of fluorescent markers the term “derivative thereof” refers to salts of the fluorophore and (or fluorophores that are conjugated to non-fluorescent moieties such as, e.g. linkers (e.g., alkyl linkers, PEG linker,) or binding moieties (e.g., maleimids, isothiocyanate or an active ester (e.g., succinimidyl ester, p-nitrophenylester, acid halogenides)) or a combination thereof.

The fluorescent markers may be linked to the probes by any means. They may be directly conjugated to the probes or conjugated via a linker. Such linker may have the length of less than 5 Å, less than 10 Å, less than 15 Å, less than 20 Å, less than 25 Å or less than 50 Å, for example. Any of the various linkers known in the art may be used (for example, as described in WO 84/03285). According to some embodiments, the linker length is between 15 and 35 Å.

According to the instant disclosure, the linkage of the markers to the probe is not negatively influencing (i.e., there is nearly no, or no significant (<2° C.) decrease in melting temperature) the melting temperature of a given probe with the target DNA. According to embodiments of the instant disclosure, the markers are attached to the nucleobase if the marker is linked to the DNA or LNA nucleotides or the marker is linked to an internucleosidic phosphate analog (such as described in WO 2007/059816). According to some embodiments, markers are attached to the 3′ or 5′ end of a probe. Such labeling methods are known in the art and commercial building blocks are available (see for example, Fluorescent oligonucleotides. Versatile tools as probes and primers for DNA and RNA analysis. Wojczewski, Christian; Stolze, Karen; Engels, Joachim W. Synlett (1999), (10), 1667-1678).

According to embodiments of the instant disclosure, the probe may be labeled with one, two, three, four, five or more fluorescent markers. According to some more specific embodiments, the probe is labeled with one or two fluorescent markers.

According to some embodiments, when the probe is labeled with two fluorescent markers, these fluorescent markers may be the fluorescent markers of the same type or different fluorescent markers. According to some more specific embodiments, the fluorescent markers labelled on the probe are different fluorescent markers. According to such embodiments, the fluorescent markers emit and absorb light of different wavelengths. In some such embodiments, the two or more fluorescent dyes form a fluorescence resonance energy transfer (FRET) pair. For example, in such embodiments the emission wavelength of the donor fluorophore may differ from that of the acceptor fluorophore in at least 25 nm, at least 50 nm, at least 100 nm, at least 150 nm, at least 200 nm or at least 250 nm, for example. Further, the emission spectrum of one fluorophore (donor) may overlap with the absorbance spectrum of the other fluorophore (acceptor).

Also, in some embodiments, the probe may also be labeled with one or more fluorophore(s) and one or more quencher(s). For example, the probe may be labeled with one fluorophore and one quencher.

According to an embodiment of the present disclosure, the probes may be hydrolysis probes additionally labeled with a quencher.

A quencher as used herein is a molecular structure that can quench the light emitted by a fluorophore. A quencher that is in a comparably near special distance to a fluorophore can decrease the light intensity emitted by the fluorophore upon excitation. Also, according to the instant disclosure, a fluorophore can, under certain circumstances, serve as a quencher.

For example, according to the instant disclosure, the quencher may quench the light upon a spatial distance of less than 10, less than 9, less than 8, less than 7, less than 6, less than 5, less than 4, less than 3 or less than 2 nucleotides between the fluorophore and the quencher.

As used in the context of the present disclosure, the terms “quencher” and “dark quencher” may be understood interchangeably. A quencher may be any molecular structure that can efficiently decrease the intensity of the fluorescence emitted by the fluorophore. A quencher may be a fluorophore or a molecular structure not emitting visible light, such as e.g., Dabsyl (dimethylaminoazosulfonic acid), a Black Hole Quencher that quenches across the entire visible spectrum, an IRDye QC-1, a Qxl quencher, an Iowa black FQ that quenches in the green-yellow part of the spectrum, or an Iowa black RQ that quenches in the orange-red part of the spectrum. The quencher may emit thermal radiation.

According to some embodiments of the instant disclosure, in the sets of probes, the probes may be 7 (instead of 8) nucleotides in length. The probe and its application are as detailed above in the context of the compositions, methods and libraries of the disclosure, wherein the two or three LNA random positions of the probes being 8 nucleotides in length is reduced to one or two LNA random position(s), respectively, in the probes being 7 nucleotides in length by deleting one random position. The set of probes may be designed as described above considering the deletion of one random position. As mentioned above, according to some embodiments, the random nucleotides may be located at the 3′ terminus.

Additionally, in some embodiments of the instant disclosure, in the sets of probes, the probes may be 9, 10, 11, 12, 13, 14, or 15 (instead of 8) nucleotides in length. The probe and its application are as detailed above in the context of the compositions, methods and libraries of the disclosure, wherein the number LNA random positions of the probes is increased in comparison to the probes being 8 nucleotides in length by introducing one or more additional random position(s), for example at least one additional random position. According to some embodiments of the present disclosure, at least one additional random position is introduced for each nucleotide exceeding 8 (e.g. two additional random positions for a probe of 10 nucleotides). The set of probes may be designed as described above considering the addition of one or more random position(s). As mentioned above, according to some embodiments, the random nucleotides may be located at the 3′ terminus.

Alternatively, and as discussed above, the term “nucleotide” may also comprise another nucleobase other than A, T, C or G, for example, uracil (U) or methyl cytosine (mC), conjugated to a molecular moiety that can polymerize. Therefore, the term “nucleotide” may alternatively refer to a ribonucleic acid (RNA) nucleotide, a nucleic acid analogue nucleotide for example, a peptide nucleic acid (PNA) nucleotide, a Morpholino nucleotide, glycol nucleic acid (GNA) nucleotide, a threose nucleic acid (TNA) nucleotide or a methylated DNA nucleotide. A nucleic acid analogue nucleotide may be selected in such a manner that it is capable of forming a stable and specific base pair with a natural base.

Alternatively, the locked nucleic acid (LNA) may also be a part of a strand comprising at least one LNA nucleotide and at least one DNA nucleotide and further comprising one of the following: a ribonucleic acid (RNA) nucleotide, a peptide nucleic acid (PNA), a Morpholino, a glycol nucleic acid (GNA), threose nucleic acid (TNA), ENA (2′-O,4′-C-ethylene-bridges nucleic acid) and 2′-amino-LNA derivatives. Alternatively, said at least one DNA nucleotide may also be replaced by one of the aforementioned nucleotides. Alternatively, said at least one LNA nucleotide may also be replaced by one of the aforementioned nucleotides.

In another aspect, the present disclosure relates to a method of determining the genotype at a locus of interest in a sample obtained from a subject, the method comprising
a) contacting the sample comprising genetic material with the composition of the present disclosure; and
b) detecting the binding of a probe of the first or the second set of probes to the genetic material, thereby determining the genotype at the locus.

As used herein, the term “determining the genotype” refers to the analysis of the genotype of an organism or a virus. Determining the genotype may include, but is not to be limited to the analysis of an allele (e.g., the analysis whether the organism has a point mutation in a certain locus or not, and which nucleotide or nucleobase has been replaced by which other nucleotide or nucleobase), the search for novel point mutation(s) in the genome of an organism or virus, or the determination or assessment of the copy number of a gene or a part of a gene.

In the context of the present disclosure, the term “locus of interest” may be understood interchangeably with the term “locus” in the broadest sense as a position in the nucleotide sequence of a gene, in particular the target gene to which the probe of the disclosure may bind. The locus of interest may comprise one, two, three, four, five, six, seven or eight nucleotide(s), for example. Also, it should be understood that there may be a mutation localized at the locus of interest, or there may be no mutation localized at the locus of interest. As such, there may also be a single nucleotide polymorphism (SNP) localized at the locus of interest, or there may be no single nucleotide polymorphism (SNP) at the locus of interest.

The term “subject” as used herein may be understood in the broadest sense as a source of genetic material. The genetic material may be obtained from any organic material, in particular a biological sample. The biological sample may be a living or dead organism, such as a living or dead bacterium, a living or dead animal, a living or dead human, a living or dead fungus or a living or dead plant, a part of an animal, a plant, a fungus, or a cell organelle a virus, a virus-like particle such as a mitochondrium, a leucoplast or a chloroplast. Additionally, according to some embodiments of the instant disclosure, DNA or RNA may be obtained from an expulsion, a scale off or a degradation product of the aforementioned organisms. For example, it may be obtained from a blood sample, an embryo blood sample, a fetal blood sample, a lymph sample, a cord blood sample, a liquor cerebrospinalis sample, an amniotic liquor/fluid sample, a mouth swab, a vaginal swab, a smear test, a dander, a hair follicle, one or more extracted cells, a sperm sample, an ovule cell, a saliva sample, a urine sample, a stool sample, a lymph sample, a sanies sample, a umbilical cord sample, a skin sample, a bone marrow sample, a mucosa sample, a tissue sample, a sample of water, a sample of soil, a sample of sediment, or a crime scene.

The term “genetic material” may refer to any kind of natural or synthetical nucleic acid that conveys or encodes genetic information by a sequence of nucleobases. The genetic material may be DNA or RNA. DNA may be double-stranded DNA or single stranded DNA. RNA may be any kind of RNA known in the art such as, e.g., mRNA, tRNA, ribosomal RNA, viral RNA, miRNA sRNA, RNAi, snRNA. The genetic material may be obtained from a biological sample or may be synthesized by organic synthesis. As used herein, the term “sample” refers to any kind of material that is analyzed by means of the probe of the present disclosure. The sample may comprise the genetic material of interest.

According to the instant disclosure, the sample may be a biological sample selected from the group consisting of a body fluid, blood, urine, serum, mucosa, sputum feces, epidermal sample, skin, cheek swab, sperm, amniotic fluid, cultured cells and bone marrow, for example. Further, according to the instant disclosure the genetic material may also be obtained by chemical synthesis as known in the art. It may be part of a naturally occurring genome or a genome of a genetically modified organism. It may be linear or circular genetic material, such as genomic DNA or a plasmid. The DNA or RNA material may also be purified, for example, by means known in the art.

The sample may be obtained from an animal. The animal may be any kind of animal, including unicellular animals (protozoa) and multicellular animals (metazoa). According to some embodiments, the animal may be a mammal, including humans. The term “animal” may also include spores or cysts of a protozoan and cysts and gametes (germ cells) of a metazoan.

Alternatively, according to the instant disclosure, the sample may be obtained from a plant. The plant may be any kind of plant, including unicellular and multicellular plants. Preferably, the plant is a useful plant, such as an agricultural crop. The term “animal,” as discussed above, may also include spores of a protozoan and seeds, fruit, fallen leaves, pollen, sap, gametes (germ cells) of a metazoan plant.

Further, according to the instant disclosure, the sample may be obtained from a bacterium. The bacterium may be any kind of protozoa, including eubacteria and archaebacteria. The bacterium may or may not be pathogenic. According to some embodiments in which the sample is a bacterium, the bacterium is a pathogenic bacterium. The term “bacterium” may also include spores of a bacterium.

Alternatively, according to the instant disclosure, the sample may be obtained from any kind of virus. The virus may also include animal and plant viruses and phages. The virus may be a virus particle (virion). Also, in some embodiments of the instant disclosure, the virus DNA and/or RNA may be obtained from virus genome that is present in a eukaryotic or a prokaryotic cell (host cell). The virus genome may be integrated in the host cell's genome. According to some embodiments in which the sample is obtained from a virus, the virus is a pathogenic virus.

Even further, according to some embodiments of the instant disclosure the sample may be obtained from a fungus. The fungus may be any kind of fungus. According to some more specific embodiments in which the same is obtained from a fungus, the fungus is a pathogenic fungus, parasiting in or on the surface of a host organism, in particular in or on the surface of an animal, including humans, or a plant. The term “fungus” may also include spores of a fungus.

Additionally, according to the instant disclosure, the DNA or RNA obtained from a biological sample may be comprised in a crude biological mixture or may be isolated. The term “crude biological mixture” may include, but may not be limited to a cell lysate that may be obtained by any means known in the art (such as lysis by means of a hypotonic buffer, detergent(s), sonication, alcohol, shear forces (e.g., by means of a French Press, a Potter or Downs homogenisator, rough pipetting), enzymes, scratching or freeze-thaw-cycle(s) (also known as, “freeze-and-squeeze”), for example) and DNA- or RNA-containing smear. The term “isolated” may also refer to dried or freeze-dried sample that, apart from salts, contains more than 25%, more than 50%, more than 60%, more than 70%, more than 80%, more than 90%, more than 95% or even more than 97% by weight DNA and/or RNA, or an aqueous or organic solution, wherein at least 25%, at least 50%, at least 60%, at least 70%, at least 80%, at least 90%, at least 95% or even at least 97% by weight of the organic content, apart from the solvent and salts, refers to DNA and/or RNA.

The DNA or RNA may be isolated by any means known in the art, such as precipitation in alcohol (e.g., ethanol, isopropanol), a mixture of different alcohol(s), chromatography-based methods (e.g., anion exchange chromatography, size exclusion chromatography), gel-based methods (e.g., gel electrophoresis such as agarose gel electrophoresis and polyacrylamide gel electrophoresis (“PAGE”)), capillary electrophoresis (CE) or combinations of two or more thereof. Several purification methods may be combined on-line or may be combined by two or more subsequent purification steps. As used herein, the terms “isolation” and “purification” may be understood interchangeably.

The DNA or RNA may also be of synthetic origin. Also, a control sample may be of synthetic origin. For example, a control sample may be a positive or a negative control, or may be used to test the selectivity of the probes. Synthetic DNA may also be linear or circular. Target DNA or target RNA of synthetic origin may be labeled by any means, in particular by means described for the probes herein. The synthetic DNA may be double-stranded or single-stranded. It may also be conjugated to a surface, such as to the surface of a bead or the surface of a plastic, glass or metal slide. According to the instant disclosure, the DNA or RNA may also be part of an array, for example, a microarray such as a DNA microarray (exemplary embodiments of DNA microarrays include a SNP array or an Affymetrix chip, and array comparative genomic hybridization) as known in the art.

According to the instant disclosure, the target sequence may be part of a target DNA or of a target RNA. The target sequence may be freely chosen. For instance, the sequence of the determined nucleotides may be known by the person skilled in the art or be obtained from a sequence database as known by those skilled in the art, such as the Ensembl Sequence Identifier, the RefSeq, the EMBL Sequence Identifier, the EMBL and/or the NCBI Entrez Genome database, or the like. Additionally, the probe design may be computer-assisted, for example with the web-based ProbeFinder Assay Design Software or LightCycler Primer Design Software (both from Roche).

The term “target DNA” as used herein may be understood as the DNA that is at least, in part, complementary to the probe of the instant disclosure. Therefore, under certain conditions, the probe can bind to the “target DNA”. It may be understood that the probe binds to complementary single strand DNA. A single strand DNA may be obtained by means known in the art, such as melting a double-stranded DNA strand as described further herein. As noted herein, “target DNA” is not required to be perfectly complementary.

According to the instant disclosure, the target DNA may be of any length. For example, the target DNA may be at least 8 nucleotides in length. The target DNA may be more than 8, more than 9, more than 10, more than 20, more than 50, more than 10², more than 10³, more than 10⁴, more than 10⁵, more than 10⁶, more than 10⁷, more than 10⁸, more than 10⁹, more than 10¹⁰ or even more nucleotides in length. It should be understood that, in the context of double-stranded DNA, the term “nucleotides in length” may also refer to the length in base pairs (bp).

Additionally, according to the instant disclosure, the target RNA may be of any length. For example, the target RNA may be at least 8 nucleotides in length. Additionally, the target RNA may be more than 8, more than 9, more than 10, more than 15, more than 20, more than 30, more than 50, more than 75, more than 100, more than 150, more than 200, more than 300, more than 500, more than 1000, more than 2000 or even more nucleotides in length. It should be understood that, in the context of double-stranded RNA, the term “nucleotides in length” may also refer to the length in base pairs (bp).

Further, it is within the scope of the present disclosure that the target DNA may be amplified by any means known in the art, such as polymerase chain reaction (PCR), amplification in bacteria, in yeast, in mammalian cells or in insect cells. Further, it is also within the scope of the present disclosure that the DNA may be obtained from a reverse transcriptase reaction, such as reverse transcriptase polymerase chain reaction. Reverse transciptase-PCR may also be used to analyze the transcriptome or a particular messenger RNA (mRNA).

According to the instant disclosure, the set of probes may be designed to characterize the genotype of the target gene. For example, one set of probes may have a stronger binding affinity to a locus of a wildtype genotype than the mutated genotype, whereas a corresponding set of probes may have a stronger binding affinity to the locus of the mutated genotype. Therefore both set of probes bind preferably to different alleles of the same locus.

Herein, the term “corresponding set of probes” refers to a probe that has an identical nucleotide sequence with the exception of the base(s) at one, two, or three LNA random position(s) and the base at the discriminating position than the probe it corresponds to.

As used in the context of the present disclosure, the term “contacting” may be understood in the broadest sense as the exposure of one or more sets of probes to the sample and vice versa. The contacting may be achieved, e.g., by ad mixing a solution comprising the one or more sets of probes to a sample. Optionally, contacting may be accompanied by a heating step.

As used throughout the disclosure, the term “detecting” may be understood in the broadest sense as the measurement of a signal occurring from a probe. The probe may be labeled with a marker and a signal occurring from the marker may be detected. Therefore, for example, the fluorescence signal occurring from an excited fluorescently labeled probe may be detected. In some embodiments, the fluorescence resonance energy transfer (FRET) signal occurring from a doubly labeled probe, the radioactive radiation occurring from a radioactively labeled probe, the melting temperature of the probe, the fluorescence depolarization of a fluorescently labeled probe, and/or the diffusion speed of a fluorescently labeled probe may be detected. Also, in some embodiments, a FRET signal and/or a fluorescence cross-correlation (FCCS) signal of two fluorescent labeled probes may be detected. These probes may interact with each other or with one target nucleotide molecule or may interact with two complementary DNA strands. Further, in some embodiments the fluorescence quenching may be detected by the loss, decrease or absence of fluorescence.

According to the present disclosure, the binding of the first set of probes may be compared to the binding of the second set of probes. In such embodiments, after contacting the sets of probes with a sample, the intensity of the signal occurring from the first set of probes may be detected. Likewise, the signal occurring from the second set of probes may be detected. The signal intensities may be compared with one another.

Further, according to the instant disclosure the first and the second set of probes can be labeled differently. The signal occurring from both sets of probes may be detected contemporaneously. The signal occurring from each one marker may be detected separately. For example, both sets of probes may be labeled with two different fluorophores emitting light of different wavelengths. The emitted light may be detected independently from another by any means known in the art, such as by separate fluorescence detectors (e.g., photomultiplier tubes (PMTS), avalanche photodiodes (APDs)), or may be detected by a single detector, but the light of the different wavelength is separated from another by one or more filter(s), one or more dichroic mirror(s), one or more prism(s), or a Meta detector, for example.

Also, as discussed herein, according to some embodiments both sets of probes may be labeled by the same marker, such as the same fluorophore. In such embodiments the signal occurring from both sets of probes may be detected subsequently.

As mentioned herein, according to some embodiments, the first and the second set of probes may differ in the discriminating position. For example, the first and the second set of probes may differ in a single nucleotide only. However, due to the difference in their nucleotide sequence, the two set of probes may have different binding affinity when binding to their target sequence. For example, the probe having a full match to the complementary nucleotide strand may have a higher affinity than the probe bearing a mismatch. According to some embodiments, one of the probes of one set of probes may show a fulmatch with the target strand, thus all nucleobases of the probe form base pairs with the nucleobases of the target strand, whereas the probes of the second set of probes bear at least a single mismatch.

According to the instant disclosure, a mismatch may lead to a comparably lower binding affinity than a fulmatch. Thus, in an equilibrium, a larger fraction of the set of probes bearing the sequence that fits better will bind. Therefore, binding of the probe showing fulmatch can be identified by obtaining an altered detection signal from the probe showing binding.

However, the person skilled in the art will notice that the target nucleotide may also have a different sequence at a certain locus.

Therefore, as described herein, one of the sets of probes may be designed to have a preference for binding to a specific genotype at the locus, whereas the other set of probes may have a preference for another genotype at the locus. Therefore, when comparing the detection signal of both probes, the genotype at the locus may be determined.

As used herein, the term “determining the genotype at the locus” may refer to the discriminating of different genotypes in a locus of interest in two or more different samples. Alternatively, the term “determining the genotype at the locus” may refer to the identification of the genotype of one sample. Further, the term “determining the genotype at the locus” may also refer to the identification of novel genotype variants in the genome of a subject.

For instance, the genotype of a subject, in particular a patient, may be determined by the method of the present disclosure to select a certain therapy. The determination of genotypes by the method of the present disclosure may also be used for epidemiologic screenings of a population. Further, the determination of a genotype by the method of the present disclosure may also be used for disease provision of a subject, for detecting a certain plant species or strain, for detecting a certain bacterial species or strain, for detecting a certain viral species or strain, for detecting a certain fungal species or strain, for detecting a certain animal species, strain, race or breed, or for criminal investigations. Further, the determination of a genotype by the method of the present disclosure may also be used for the detection of genotypic characteristics of animals, plant, viruses, bacteria and/or fungi. As known in the art, genotypic characteristics may have influences on the phenotype. Therefore, the method may be used to predict phenotypic characteristics. Further, the method may be used to detect a predisposition of a disease in an animal, including human, or a plant, or a fungus. Further, the method of the present disclosure may be used for prenatal diagnostics. According to the instant disclosure, the sample may be obtained from the blood or the lymph of the embryo or fetus, the cord blood, the placenta or the amniotic liquor/fluid.

According to an embodiment of the present disclosure, the locus is a single nucleotide.

For example, a single nucleotide in a particular position of the target DNA or target RNA may be of interest. Therefore, there may be a single nucleotide polymorphism (SNP) in the locus.

According to a further embodiment, the method may comprise:
performing an amplifying step comprising contacting the sample with a set of primers to produce an amplification product including the locus of interest,
performing a hybridizing step comprising contacting the amplification product of step a) with the composition of the present disclosure; and
detecting the hybridizing of a probe of the first or the second set of probes to the genetic material, thereby determining the genotype at the locus.

The term “amplifying step” as used herein refers to any method known in the art for amplifying genetic material. DNA may be amplified, for example, by using polymerase chain reaction, may be amplified in cells (e.g., in bacteria cells, in mammalian cells, in insect cells). RNA may be amplified by using reverse transcriptase PCR.

The term “contacting the sample with a set of primers” refers to the addition of primers to the sample comprising the target DNA. Further enzyme(s) such as DNA polymerase, magnesium salt(s), nucleotides triphosphates (dATP, dCTP, dTTP, dGTP) and/or a suitable buffer may be added. These ingredients are known in the art and, at least in some cases, commercially available.

The term “hybridizing step” may refer to any method known in the art for hybridizing the probe with its target DNA or target RNA. The probes may hybridize with their target DNA or target RNA under conditions a short nucleic acid regularly hybridizes with its target sequence. Such conditions are generally well-known in the art.

For hybridization, double-stranded DNA may be first denaturated, thus, both DNA strands may be separated from another. For example, the target DNA may be denaturated by heating the sample. The target DNA strand may be denaturated at more than 40° C., more than 50° C. more than 60° C., more than 70° C., more than 80° C., more than 90° C., more than 95° C., more than 96° C., more than 97° C., more than 98° C. or even more than 99° C. According to a specific embodiment, the target DNA strand may be denaturated at more than 60° C., more than 70° C., more than 80° C., more than 90° C., more than 95° C., more than 96° C., more than 97° C., more than 98° C. or even more than 99° C. By denaturating the DNA strand, the two strands on the double helix are separated. This process may be also designated as “melting” of DNA.

In a solution comprising the target DNA and the respective probe as used in the present disclosure, the probe may anneal with the DNA strand when cooling said solution down to less than 75° C., less than 70° C., less than 65° C., less than 60° C., less than 55° C., less than 50° C., less than 45° C., less than 40° C., less than 39° C., less than 38° C. or even less than 37° C.

The term “amplification product” may be understood as the product of the amplification step as described herein. Typically, the amplification product is shorter than the target DNA, but longer than the probe. The locus of interest may be included in the amplification product.

The probe may also be used for a polymerase chain reaction (PCR) as known in the art. Herein, the probes may be labeled or unlabelled. According to some embodiments, both probes are labeled. The two probes may be labeled with two different fluorophores for example. Alternatively, one probe may be labeled with a fluorophore and the other probe may be labeled with a quencher. In some embodiments, one probe may also be labeled with two different fluorophores, or with one fluorophore and one quencher. The probe(s) and/or primer(s) may be comprised in a solution further comprising the target DNA, a buffer (comprising water and optionally comprising a pH buffer, a magnesium salt and cofactors of the DNA polymerase), DNA polymerase, a nucleotide mixture (comprising dATP, dGTP, dCTP and dTTP).

According to embodiments of the instant disclosure, the target DNA strand is denaturated. For instance, depending on its length, a target DNA strand may be denaturated as described above. By denaturating the DNA strand, the two strands on the double helix separate.

Following denaturation of the target DNA strand, the solution is cooled down to less than 75° C., less than 70° C., less than 65° C., less than 60° C., less than 55° C., less than 50° C., less than 45° C., less than 40° C., less than 39° C., less than 38° C. or even less than 37° C.

Thereafter, the DNA strand may then be elongated, for example, at the temperature optimum of the utilized DNA polymerase. Such temperature may be in the range of 50 to 80° C. or in the range of 50 to 65° C., for example. Then the next cycle may start by denaturation of the DNA strands. Further, according to the instant disclosure, detection may be in real-time.

It is within the scope of the present disclosure that the PCR reaction may be conducted by any means known in the art. For example, it may be conducted manually or automatized. Automatized PCR may be conducted on a standard PCR machine or on a real-time PCR machine (e.g. a LightCycler®), for example. Further, the PCR may be quantitative PCR (qPCR) and/or may be reverse transcriptase PCR(RT-PCR). Different PCR methods may also be combined with another. The PCR reaction may be combined by detection of a FRET effect, for example, which may be in real-time.

According to illustrative embodiments of the present disclosure, the method is characterized in that,
(i) the detecting is by measuring presence or absence of fluorescence;
(ii) the detecting is in real-time;
(iii) the marker is selected from the group consisting of fluorescein, LC-Yellow 555, FAM, VIC, HEX, Rhodamine B, Rhodamine 6G, LC-Red 610, LC-Red 640, LC-Red 670, LC-Red 705, Cy3, Cy3.5, Cy5, Cy5.5 and a quencher;
(iv) the amplifying step employs a polymerase enzyme having 5′ to 3′ exonuclease activity; and/or
(v) the sample is a biological sample, for example a sample selected from the group consisting of a body fluid, a blood sample, a urine sample, serum, mucosa, sputum feces, epidermal sample, skin sample, cheek swab, sperm, amniotic fluid, cultured cells and bone marrow sample.

According to the instant disclosure, the probe may be labeled with a marker, in particular a fluorescent marker such as, e.g., fluorescein dyes (e.g., fluorescein, fluorescein isothiocyanate (FITC), rhodamine dyes (Rhodamine, Rhodamine B, Rhodamine 6G, tetramethylrhodamine (TAMRA), rhodamine isothiocyanate) cyanine dyes (e.g., Cy3, Cy3.5, Cy5, Cy5.5, Cy7), LC dyes (e.g., LC-Yellow 555, LC-Red 610, LC-Red 640, LC-Red 670, LC-Red 705), Alexa dyes (Alexa 488, Alexa 546, Alexa 647), S0387, HOECHST dye, erythrosine isothiocyanate, Oregon Green, Lucifer Yellow, VS, phycoerythrin, FAM, LightCycler® Yellow 555, VIC, HEX or quantum dots.

The fluorophore may be excited with light of a wavelength near to one of its absorbance maxima. The fluorophore may also be excited by light of a high intensity of the double wavelength of one of its absorbance maxima (two photon effect).

The probe may be labeled with one, two, three, four, five or more fluorescent markers. According to some embodiments, the probe is labeled with one or two fluorescent markers.

According to embodiments of the present disclosure, in which the probes are labeled with two fluorescent markers, such fluorescent markers may be the fluorescent markers of the same type or may be different fluorescent markers. Further, when the probe is labeled with two fluorescent markers, the emission spectrum of donor fluorophore may overlap with the absorbance spectrum of the acceptor fluorophore. Also, in embodiments when the probe is labelled with two fluorescent markers, the two fluorophores may enable FRET (fluorescence resonance energy transfer).

According to some embodiments of the present disclosure in which the probe includes two fluorophores, the donor fluorophore may absorb light at a shorter wavelength than the acceptor fluorophore. Further, the donor fluorophore may emit light at a shorter wavelength than the acceptor fluorophore. Additionally, the emission spectrum of the donor fluorophore may largely overlaps with the absorbance spectrum of the acceptor fluorophore. Alternatively, it is also possible that the maximum of the excitation spectrum of the donor fluorophore may be at the double wavelength of the absorbance maximum of the acceptor fluorophore. For example, then a two-photon system may be used for FRET.

The FRET technology is well-known in the art (see for example U.S. Pat. Nos. 4,996,143, 5,565,322, 5,849,489, and 6,162,603). It is based on a concept that energy is transferred from a donor fluorophore to an acceptor fluorophore that on its part emits light. Upon irradiation of the donor fluorophore with a certain wavelength that is absorbed by the donor fluorophore the donor fluorophore is excited. If no acceptor fluorophore is in its near special distance, the donor fluorophore emits light of a certain red-shifted wavelength (bathochrome effect). But, if an acceptor fluorophore is in its near special distance, FRET occurs. The energy transfers from the donor fluorophore to the acceptor fluorophore occurs by resonance energy transfer (Förster energy transfer), thus, preferably without emitting light. Hereby, the acceptor fluorophore is excited and may emit light. The light emitted by the acceptor fluorophore is further red-shifted (bathochrome shift, Stokes shift). The stokes shift of the used fluorophores may be more than 20 nm, more than 30 nm, more than 40 nm, more than 50 nm, more than 75 nm, more than 100 nm, more than 125 nm, more than 150 nm or more than 200 nm, for example.

The occurrence of a FRET effect can be determined and quantified in different ways. The decrease of the light intensity emitted by the donor fluorophore upon the occurrence of FRET may be quantified. Alternatively or additionally, the increase of the light intensity emitted by the acceptor fluorophore upon the occurrence of FRET may be quantified.

The FRET intensity depends on the special distance between the donor and the acceptor fluorophore. The spatial distance on the probe may be less than 10, less than 9, less than 8, less than 8, less than 7, less than 6, less than 5, less than 4, less than 3, or even less than 2 nucleotides. For example, the spatial distance between the donor and the acceptor fluorophore may be within the Förster radius.

The donor fluorophore may be excited with any kind of light source. The light source emits light of a defined wavelength. The light source may be, an argon ion laser, a high intensity mercury (Hg) arc lamp, an LED diode, a HeNe laser, a HeCd laser, or the like. The excitation light may be directed through one or more filter(s) and/or one or more dichroic mirror(s) selecting the desired wavelength. Likewise, the emitting light may be directed through one or more filter(s) and/or one or more dichroic mirror(s) selecting the desired wavelength.

The FRET experiment may be carried out on any experimental setup known in the art for quantifying FRET intensity. The experimental setup may comprise, e.g., a photon counting epifluorescent microscope (containing the appropriate dichroic mirror(s) and filter(s) for monitoring fluorescent emission at the particular range), a photon counting photomultiplier system or photometer.

Detecting the FRET may be conducted by measuring presence or absence of fluorescence. As used in the context of the present disclosure, the term “absence of fluorescence” refers to a fluorescence rate of than 20%, less than 15%, less than 10%, less than 5%, less than 4%, less than 3%, less than 2%, less than 1% or even less than 0.5% of the fluorescence intensity that is maximally found for the same fluorophore under the same conditions (such as in the same buffer, at same temperature, with the same excitation intensity on the same apparatus with the same settings).

For an intact probe, a strong FRET effect may be detectable. For a cleaved probe or a probe of which one or two fluorophores were cleaved off, no or a far lower FRET effect may be detectable. Therefore a FRET probe may be a hydrolysis probe, such as, e.g., a TaqMan® probe, as described herein.

Alternatively, the occurrence of two fluorophores on one molecular structure may also be determined by fluorescence cross-correlation spectroscopy (FCCS). Herein, it may be determined whether the fluorescence signals occurring from two different fluorophores of a doubly labeled probe diffuse conjointly or independently. Alternatively, it may be determined whether the fluorescence occurring from two different fluorophores of two differently labeled probes binding to the same target DNA diffuse conjointly or independently.

Further, an amplified luminescence proximity homogenous assay (ALPHA, AlphaScreen) may be used.

The probes can also be labeled with one fluorophore only. In such embodiments, the binding to the target sequence may be detected by measuring the fluorescence depolarization or the diffusion speed. The term “diffusion speed” may include the lateral and tangential diffusion velocity, the rotational speed of the molecule, the intramolecular rotational speed, any kind of molecular oscillation and combinations thereof.

Flourescence depolarization bases on fluorescence anisotropy. In fluorescence depolarization assays the rotational diffusion of a molecule is determined from the decorrelation of polarization in fluorescence, i.e., between the exciting and emitted (fluorescent) photons. This decorrelation may be measured as the “tumbling time” of the molecule as a whole, or of a part of the molecule relative to the whole. From the rotational diffusion constants, the experimenter may determine whether fast tumbling low-molecular weight probe has bound to its slowly tumbling high-molecular weight target sequence.

The diffusion speed may be measured by fluorescence correlation spectroscopy (FCS) as known in the art. Herein, the dwelling time of freely diffusion molecules is measured. From the average dwelling time (diffusion constant), the experimenter may determine whether fast diffusing low-molecular weight probe has bound to its slowly diffusing high-molecular weight target sequence or the low-molecular weight probe is diffusing freely.

The probe may also be labeled with one or more fluorophore(s) and one or more quencher(s). For example, the probe may be labeled with one fluorophore and one quencher.

In another embodiment of the present disclosure, the probes may be hydrolysis probes additionally labeled with a quencher.

A quencher as user herein is a molecular structure that can quench the light emitted by a fluorophore. Therefore, a quencher that is in a comparably near special distance to a fluorophore can decrease the light intensity emitted by the fluorophore upon excitation.

The quencher may quench the light upon a spatial distance, for example, of less than 10, less than 9, less than 8, less than 7, less than 6, less than 5, less than 4, less than 3 or less than 2 nucleotides between the fluorophore and the quencher.

The probe can also be a TaqMan® probe, a molecular beacon, a scorpion primer or a lux primer as known in the art. According to some embodiments, the probe is a TaqMan® probe.

The probes may also be labeled with a non-fluorescent dye such as a p-nitrophenyl moiety or Malachite Green, or with reactive small molecules that can bind to other molecules such as maleimides, isothiocyanates or active esters (e.g., succinimidyl esters, p-nitrophenyl esters). Further, the probes may be labeled with small molecules selectively binding to high-molecular weight molecules such as biotin methotrexate or glycocorticoids. A probe labeled with biotin may be detected by using labeled strepavidine, for example. A probe labeled with methotrexate may be detected by using labeled dihydrofolate reductase (DHFR), for example. A probe labeled with a glycocorticoid may be detected by using labeled a labeled antibody or antibody derivatives (such as Fab fragments, single chain antibodies, diabodies, triabodies, and tandabs) directed against said glycocorticoids, for example. Alternatively, the probe may be unlabeled and may be detected by a labeled antibody or a labeled antibody derivative (e.g., a Fab fragment, a single chain antibody, a diabody, a triabody, a tandab). Further, the probe may be unlabeled and may be detected by an unlabeled antibody or an unlabeled antibody derivative (e.g., a Fab fragment, a single chain antibody, a diabody, a triabody, a tandab) that is on its part detected by a labeled antibody or antibody derivative directed against its Fc part.

Further, according to the instant disclosure the probe may be conjugated with an enzyme that can generate a color from a precursor (e.g., a peroxidase, alkaline phosphatase). This conjugation may be obtained by conjugating the probe covalently to the enzyme or by conjugating the probe with a binding molecule (e.g., digoxigenine, methotrexate) that can bind to a fusion protein of the enzyme enzyme that can generate a color from a precursor and a protein binding to the binding molecule. Alternatively, the enzyme may be able to emit light by chemical conversion (chemoluminescence) (e.g., luciferase).

Alternatively, the probes may be radioactively labeled. Therefore, the probe may be labeled with ³H, ³²P, ³⁵S, ¹⁴C lanthonoids or other radioactive labels.

Alternatively, the probes may be labeled with heavy atoms detectable by nuclear magnetic resonance (NMR) or mass-spectrometry (e.g., ESI− or MALDI-MS). Therefore, the probe may be labeled with ²H or ¹³C, for example.

Moreover, the melting point of the probe(s) may be determined. For example, a higher melting point may refer to a stronger binding. Thus, a probe binding stronger to its target sequence than a corresponding probe bearing one or more altered nucleotide positions has lesser mismatches with the target sequence than the corresponding probe. Thereby the genotype of one or more samples may be characterized according to an embodiment of the instant disclosure.

According to an embodiment of the instant disclosure, one of the probes of one of the two or more sets of probes may show a fulmatch with the target sequence, the other probes of the same set of probes may show one or more mismatch(es) with the target sequence. The probes of the corresponding set(s) of probes may show an additional mismatch in the discriminating position.

Further, according to the instant disclosure, the probes may be TaqMan® probes. TaqMan® probes may be used to conduct a TaqMan® assay, for example, as known in the art. As used herein, the terms “TaqMan® probe” and “hydrolysis probe” may be understood interchangeably. The TaqMan® probe comprises a fluorophore and a quencher. In some embodiments, the fluorophore and the quencher are located near the termini of the probe, and in some such embodiments, the fluorophore is located near the 5′ terminus and the quencher is located near the 3′ terminus. The term “3′-terminal” may be understood in the broadest sense as understood in the art. Further, the terms “3′ terminus” and “3′ end” may be understood interchangeably as known in the art. Also, it should be understood that the terms “3′ terminus” and “3′ end” as used herein may refer to the 5′ end of the nucleotide strand, but may not exclude that at the 3′ end another molecular moiety (such as, e.g., a fluorophore, a quencher, a binding moiety or the like) is added to the 3′ end of the probe.

As used herein, the term “located near” generally means that the fluorescence or quencher moiety is located not more than 4, not more than 3, not more than 2, and even at the first nucleotide from the respective terminus.

The TaqMan® probe may hybridize to its target sequence. Further, the used composition may further comprise a pair of primers, thus one forward and one reverse primer. These primers are generally unlabeled. Further, generally, the forward primer binds upstream, the reverse primer downstream of the band, such that the TaqMan® probe binds to a sequence that is a part of the strand that is amplified. A PCR reaction as well-known in the art is conducted. Thus, the target DNA is melted, then conditions are chosen that enable the annealing of the primers and the probe to the target DNA. Subsequently, conditions are chosen that enable the DNA polymerase to amplify the DNA strand between the primers. In the context of the TaqMan® assay, the DNA polymerase generally has a 5′ to 3′ exonuclease activity. Also, the DNA polymerase may be Taq polymerase or a functional variant thereof. When the DNA polymerase comes to the TaqMan® probe, the 5′ end is cleaved off. Thereby, the fluorophore or quencher bound to the 5′ terminal nucleotide(s) is also cleaved off. Preferably, the fluorophore is cleaved off. Consequently the fluorophore and the quencher may diffuse in different directions. The spatial distance between both may be significantly increased and the fluorescence occurred by the fluorophore is significantly increased as it is not quenched by the dark quencher any longer. Also, the TaqMan® assay may be analyzed in real-time. The TaqMan® assay may also be conducted during a life-time PCR method. It may also be conducted quantitatively in a qPCR reaction.

A TaqMan® assay using the probes of the present disclosure may be used for the discrimination of alleles, genotyping, bacterial identification assays, DNA quantification, and the determination of the viral load in clinical specimen, gene expression assays and verification of microarray results. It may also be used for the discrimination of alleles, genotyping, and bacterial identification assays. Genotyping may be single nucleotide polymorphisms (SNP) genotyping, for example, and therefore include the determination of a genotype at defined a locus of interest in a sample, wherein the locus is a single nucleotide. Alternatively, genotyping may be copy number variant (CNV) genotyping. A copy number variant (CNV) is a segment of DNA in which differences of copy-number (number of copies of a DNA sequence or portions thereof) have been found by comparison of two or more genomes. As discussed above, sequences (and loci of various SNPs and CNVs) may be obtained from databases such as The Database of Genomic Variants (DGV), the NCBI dbSNP database, the UCSC Genome Bioinformatics Site, the DatabasE of Chromosomal Imbalance and Phenotype in Humans using Ensembl Resources (DECIPHER), the HapMap Project, the Sanger Institute Copy Number Variation Project and the Human Structural Variation Project.

A similar assay may also be designed using two molecules that show a FRET effect instead of the fluorescence quenching. In such embodiment, one of the fluorophores is cleaved off upon a 5′ to 3′ nuclease activity of the DNA polymerase. Preferably, the DNA polymerase bears a 5′ to 3′ exonuclease activity, such as Taq polymerase. The spatial distance between the fluorophores is increased. The FRET efficiency decreases upon cleavage of fluorophore located at the 5′ end. The fluorophore located at the 5′ end may be the donor fluorophore or the acceptor fluorophore. According to some embodiment, the fluorophore located at the 5′ end may be the donor fluorophore. Also, this assay may be conducted during a life-time PCR method and it may also be conducted quantitatively in a qPCR.

Determining the genotype may be performed by a hybridization- and/or PCR-based method as described herein. For example, determining the genotype may be performed in a PCR-based method. More specifically, according to the instant disclosure, determining the genotype may be performed in a PCR— and TaqMan®-based method.

As described herein, a hybridization step with two or more probes may be performed. As described herein, the probes may each be labeled with one or more fluorophore(s), two different fluorophores, or a fluorophore and a quencher, for example. Further, the signal occurring from the probes may be detected, for example, a FRET signal (indicating the increase of fluorescence) may be detected. As described herein, according to the instant disclosure the probe may be a TaqMan® probe comprising a fluorophore and a quencher. Also, detection may be based on the presence or absence of fluorescence or on an increase or decrease of fluorescence.

Also, the probes of the present disclosure may be used for in situ hybridization, for example, as in comparative in situ hybridization and/or fluorescent in situ hybridization (FISH). Herein, the probe may be fluorescently or radioactively labeled or may be detected by an antibody or an antibody derivative that is either labeled or that can be detected by a second antibody directed against its Fc part. Further, the probe may be conjugated with an enzyme that can generate a color from a precursor (e.g., a peroxidase, alkaline phosphatase).

Further, the probes of the present disclosure may be used in other methods based on probe hybridization. The probes of the present disclosure may be used in microarray methods, for example. The probes may be used in microarray validation. Moreover, the probes may be used in gene-knockdown quantification assays.

Further, the method of the present disclosure may be used for allele-specific PCR, wherein one of the probes may serve as a primer that is combined with a suitable primer or wherein two probes serve as forward and reverse primers. Preferably, one of the probes may serve as a primer that binds at the locus where the different alleles are located or are assumed to be located.

Further, the probe of the present disclosure may be used for microarray expression profiling, small RNA research, gene repair/exon skipping, splice variant detection, DNAzymes and/or comparative genome hybridization (GCH). These methods are well-known to those skilled in the art.

A probe of the present disclosure may also be used in a method for down-regulating expression of a gene. Therefore, the probe may serve as an antisense oligonucleotide such as RNAi (e.g., sRNA, miRNA, snRNA) as known in the art. The antisense oligonucleotide may interfere with mRNA of the target organism. The target organism may be an animal, including human or a plant, for example. Antisense oligonucleotides are well-known to those skilled in the art.

The composition of the present disclosure may be combined with a computer software for analyzing the data.

In a more specific embodiment of the present disclosure, the disclosure relates to a library of at least two sets of probes, wherein the library comprises a plurality of sets of probes each of the probes having eight nucleotides with the general structure 5′-DL L L L L X X-3′ or 5′-D-L-L-L-L-X-X-X-3′, wherein D indicates a DNA nucleotide, L indicates a LNA nucleotide and X indicates a LNA random nucleotide,

wherein within one set of probes all probes have identical nucleotide sequences with the exception of the two and/or three LNA random nucleotides, wherein at each position of a LNA random nucleotide the base is independently selected from adenine, cytosine, guanine and thymine and any possible sequence resulting from the base variation(s) at the two positions is represented by a probe in each set of probes; and

wherein one set of probes differs from another set of probes in the sequence of at least the DNA nucleotide D or an LNA nucleotide L.

As used herein, the term “library” refers to a collection of several sets of probes, wherein the DNA nucleotides and the LNA nucleotides may be located in the same position in each of the probes. Further, in a library of sets of probes, the random positions and the determined positions, and the discriminating position may be located at the same position in each probe of the library.

Said library may be designed to cover a plurality of targets in the genome of one species or the genome of different species. Such a probe library may be a library of probes for any technical problem, for example.

According to embodiments of the instant disclosure, for sets of probes of which three nucleotides are random nucleotides and five nucleotides are determined, 1024 sets of probes are sufficient to provide a suitable library of probes for any technical problem. As described herein, according to the instant disclosure two sets of probes are needed, of which at least the discriminating position of each one nucleotide is known, thus a library comprising 512 sets of probes is sufficient to apply to any technical problem.

According to embodiments of the instant disclosure, for sets of probes of which two nucleotides are random nucleotides and six nucleotides are determined, 4096 sets of probes are sufficient to provide a suitable library of probes for any technical problem. As described herein, according to the instant disclosure two sets of probes are needed of which at least the discriminating position of each one nucleotide is known, thus a library comprising 2048 sets of probes is sufficient to apply to any technical problem.

In an exemplified embodiment, the library of sets of probes is characterized in that,
(i) number of sets amounts to at least 64, preferably 128, especially at least 256, particularly at least 512, more preferably at least 1024, most preferably 2048; and/or
(ii) the sets of probes are spatially separated from each other.

The term “spatially separated from each other” means that the probes may be stored separated from another, thus, e.g., in different vials or different wells. The vials may be plastic vials or glass vials, such as, e.g., a plastic cup, a screw cap vial or a sealed vial. A well may, e.g., refer to a well of a multiwell (multichamber plate, multititer plate) plate such as, e.g., an 8-well plate, 12-well plate, a 24-well plate, a 96-well plate, a 384-well plate or a 1536-well plate as known in the art. They may be stored at conditions suitable for single stranded DNA and/or LNA probes of the given length. They may be stored at room temperature, at 4° C., at −20° C., at −80° C. or in liquid nitrogen. They may be stored in water, in an aqueous buffer, in an organic solvent, such as DMSO. Further, they may be freeze-dried.

A library of said probes may comprise at least two, at least 64, at least 128, at least 256, at least 512, at least 1024, at least 2048, at least 4096 or more probes. The library of probes may comprise large parts of the genome of the organism or even the whole genome. The library of probes may comprise one, two, three, four, five or more different alleles for a particular locus of interest. There may be no, one, two, three or more loci of interest in a single gene.

The following examples, sequence listing, and figures are provided for the purpose of demonstrating various embodiments of the instant disclosure and aiding in an understanding of the present disclosure, the true scope of which is set forth in the appended claims. These examples are not intended to, and should not be understood as, limiting the scope or spirit of the instant disclosure in any way. It should also be understood that modifications can be made in the procedures set forth without departing from the spirit of the disclosure.

### EXAMPLES

### Example 1

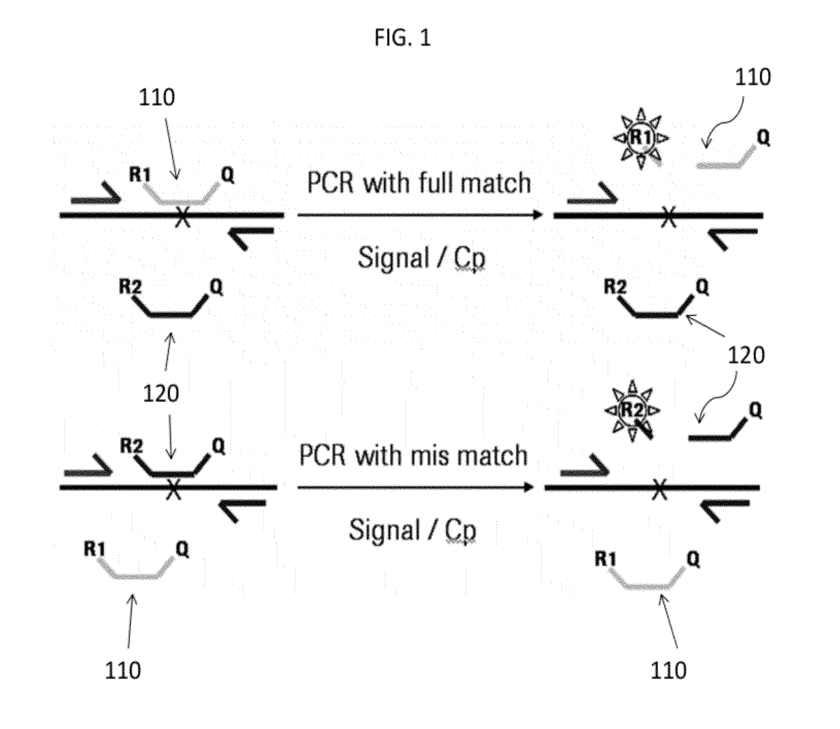

The experiment demonstrates the discriminating power of a full match probe in comparison to the mis-match probe, as disclosed herein, where the full match and the mis-match probe have the same nucleic acid sequence except one nucleic base in the middle of the probe sequence. (FIG. 1) The primer pairs are for both probes and produce the same amplicon during PCR amplification. Because both probes have the same reporter dye, the PCR experiment was performed in different wells in mono color mode only. Further, in order to demonstrate that the PCR performance is sufficient even with different sample concentrations, the PCR was performed with two different dilutions of cDNA target (assayed in duplicate for each concentration). The target parameter in example 1 is 18S.

With reference to FIG. 1, first probe 110 (having reporter 1 indicated as R1) is shown hybridized and cleaved in the presence of a full match (to the wild type sequence) in a sample allowing R1 to produce a signal (top schematic). Second probe 120 (having reporter 2 indicated as R2) is shown hybridized and cleaved in the presence of a full match (to the mutant sequence) in a sample allowing R2 to produce a signal (bottom schematic). Quencher Q is also shown comprising both first probe 110 and second probe 120. As indicated from FIG. 1, R¹ and R² produce the signal upon release of quencher, in a manner as previously described herein.

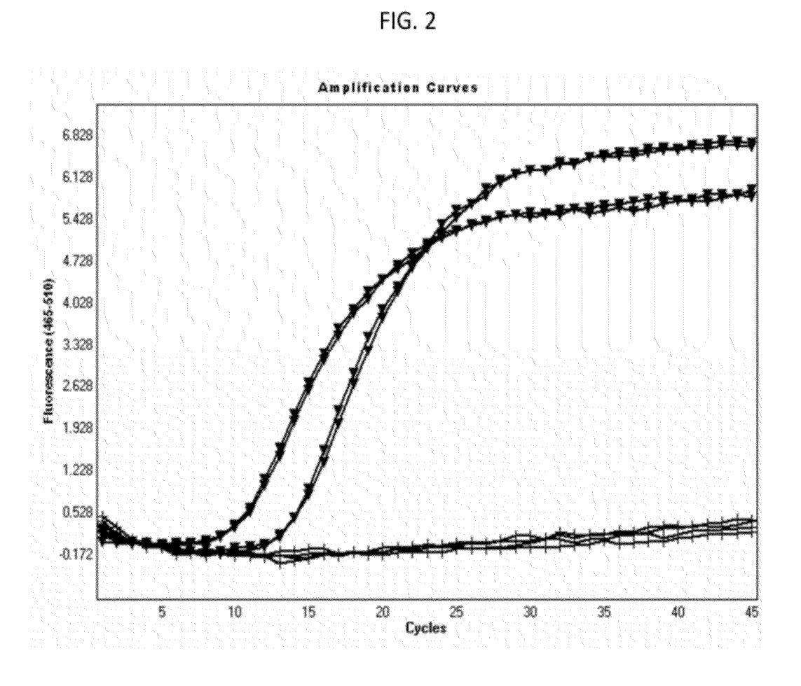

As shown in FIG. 2 the PCR reactions with a probe having a full match sequence to the target sequence (exemplified as first probe 110 in FIG. 1) show a sigmoidal amplification curve and therefore a positive Cp call while the probe having a mis match to the target sequence (exemplified as second probe 120 in FIG. 1) show no sigmoidal amplification curve and a negative Cp call. With reference to FIG. 2, the triangle style points illustrate a positive Cp call and a sigmoidal amplification curve (generated from a full match probe) at different sample concentrations. The curves having cross style points (along the bottom) illustrate negative (no Cp) calls and no sigmoidal amplification curve (generated from a mismatch probe) at different sample concentrations.

The present experiment was carried out as illustrated in FIG. 1 (with the exception, as noted above, that both probes have the same reporter), according to the following specifications:

Mono Color PCR Assay with Parameter 18S

General probe structure:
5′-D-L-L-L-L-L-X-X-3′
FAM full match probe sequence:
5′-X-t-T* [symbol] *T*T*G*-(AGCT)*(AGCT)*-Z-3′
FAM mismatch probe sequence:
5′-X-t-T* [symbol] *T*T*G*-(AGCT)*(AGCT)*-Z-3′

whereas X═FAM,
t=dT
T*, A*, C* or G*=LNA T, A, C or G
(ACCT)*=wobble bases of LNA T, A, C and G (random position)
Z=BHQ2-Quencher (Black Hole Quencher 2)
Bold and italic fonts indicate discriminating position.

(SEQ ID NO: 1)

| | |
|---|---|
| Primer forward sequence: | gacggaccagagcgaaag |

(SEQ ID NO: 2)

| | |
|---|---|
| Primer reverse sequence: | cgtcttcgaacctccgact |

PCR Cycler: LightCycler® 480 Instrument, 384-well block (Roche Applied Science, Cat. No.: 04 545 885 001)
PCR Reagents: LightCycler® 480 Probes Master (Roche Applied Science, Cat. No.: 04 707 494 001); LightCycler® 480 Multiwell Plate, 384 (Roche Applied Science, Cat. No.: 04 729 749 001)
Sample material: cDNA, reverse transcribed from RNA (Takara/Clontech, Cat. No.: 636538) with Transcriptor First Strand cDNA Synthesis Kit (Roche Applied Science, Cat. No.: 04 897 030 001). The cDNA synthesis step was performed as described in the pack insert of the corresponding kit.
Sample concentration: 50 ng and 5 ng per well in 10 μl final PCR volume
PCR protocol for Example 1: see table below

PCR Protocol for Example 1:

| | ° C. | Time | | RR(° C./s)(96) | Aq. Mode | Cycles |
|---|---|---|---|---|---|---|
| Preinkubation | 95 | 10 | min. | 4.8 (4, 4) | — | — |
| Amplifikation | 95 | 10 | s | 4.8 (4, 4) | none | 30 |
| denat. | | | | | | |
| anneal. | 37 | 30 | s | 2.5 (2, 2) | none | — |
| elong. | 50 | 30 | s | 4.8 (4, 4) | Single | — |
| Cooling | 40 | 30 | s | 2.2 (2, 5) | none | — |

### Example 2

The experiment demonstrates the discriminating power of a full match probe in comparison to a mis match probe (as depicted in FIG. 1), where the full match and the mis match probe have the same nucleic acid sequence except one nucleic base near the middle of the probe sequence. The primer pairs are for both probes, and produce the same amplicon during PCR amplification. Because both probes have the same reporter dye the PCR experiment was performed in different wells in mono color mode only. Further, in order to demonstrate that the PCR performance is sufficient even with different sample concentrations the PCR was performed with two different dilutions of cDNA target (assayed in duplicate at each concentration). The difference between example 2 compared to example 1 is the target parameters (the target parameter in example 2 is MNAT1).

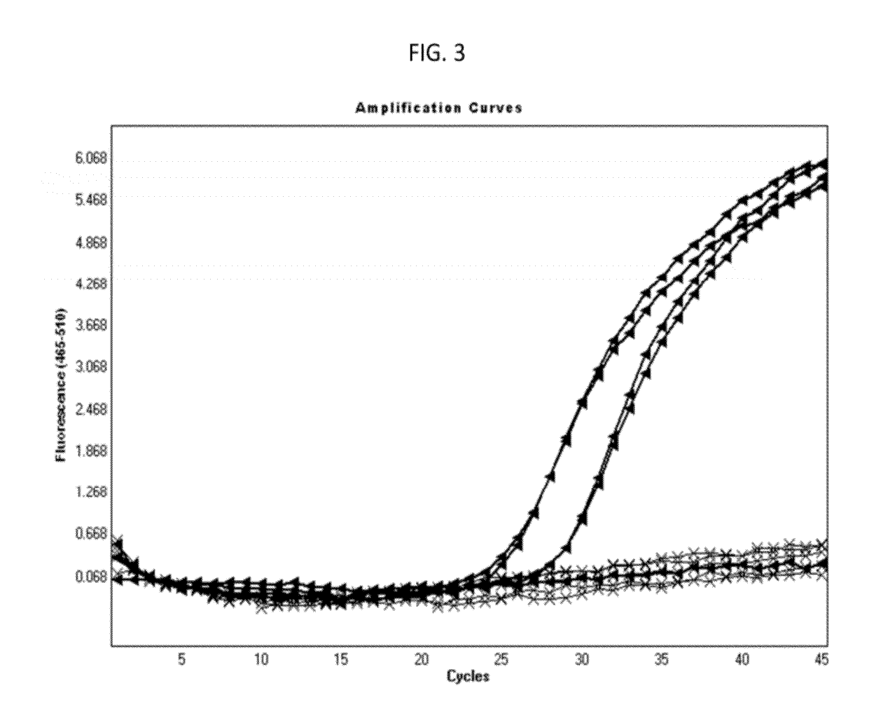

With reference to FIG. 3, the PCR reactions with a probe having a full match sequence to the target sequence (exemplified as first probe 110 in FIG. 1) show a sigmoidal amplification curve and therefore a positive Cp call while the probe having a mis match to the target sequence (exemplified as second probe 120 in FIG. 1) show no sigmoidal amplification curve and a negative Cp call. With reference to FIG. 3, the triangle style points illustrate a positive Cp call and a sigmoidal amplification curve (generated from a full match probe) at different sample concentrations. The curves having cross style points (along the bottom) illustrate negative (no Cp) calls and no sigmoidal amplification curve (generated from a mismatch probe) at different sample concentrations.

The present experiment was carried out as illustrated in FIG. 1 (with the exception, as noted above, that both probes have the same reporter), according to the following specifications:

Mono Color PCR Assay with Parameter MNAT1

General probe structure:
5′-D-L-L-L-L-L-X-X-3′
FAM full match probe sequence:
5′-X-t-T* [symbol] *A*T*G*-(AGCT)*(AGCT)*-Z-3′
FAM mismatch probe sequence:
5′-X-t-T* [symbol] *A*T*G*-(AGCT)*(AGCT)*-Z-3′

whereas X=FAM, t=dT
T*, A*, C* or G*=LNA T, A, C or G
(ACCT)*=wobble bases of LNA T, A, C and G (random position)
Z=BHQ2-Quencher (Black Hole Quencher 2)
Bold and italic fonts indicate discriminating position.

(SEQ ID NO: 3)

| | |
|---|---|
| Primer forward sequence: | cccaaacctgtaaaaccagtg |

(SEQ ID NO: 4)

| | |
|---|---|
| Primer reverse sequence: | ttgtgaataggtgccagtgaa |

PCR Cycler: LightCycler® 480 Instrument, 384-well block (Roche Applied Science, Cat. No.: 04 545 885 001)
PCR Reagents: LightCycler® 480 Probes Master (Roche Applied Science, Cat. No.: 04 707 494 001); LightCycler® 480 Multiwell Plate, 384 (Roche Applied Science, Cat. No.: 04 729 749 001)
Sample material: cDNA, reverse transcribed from RNA (Takara/Clontech, Cat. No.: 636538) with Transcriptor First Strand cDNA Synthesis Kit (Roche Applied Science, Cat. No.: 04 897 030 001). The cDNA synthesis step was performed as described in the pack insert of the corresponding kit.
Sample concentration: 50 ng and 5 ng per well in 10 μl final PCR volume

PCR Protocol for Example 2:

### FAM-Kanal:465:510

| | ° C. | Time | | RR(° C./s)(96) | Aq. Mode | Cycles |
|---|---|---|---|---|---|---|
| Preinkubation | 95 | 10 | min. | 4.8 (4, 4) | — | — |
| Amplifikation | 95 | 10 | s | 4.8 (4, 4) | none | 30 |
| denat. | | | | | | |
| anneal. | 37 | 30 | s | 2.5 (2, 2) | none | — |
| elong. | 50 | 30 | s | 4.8 (4, 4) | Single | — |
| Cooling | 40 | 30 | s | 2.2 (2, 5) | none | — |

### Example 3

The experiment was carried out to demonstrate the discriminating power of the full match probe in comparison to the mis match probe, where the full match and the mis match probe have the same nucleic acid sequence except one nucleic base in the middle of the probe sequence. The primer pairs are for both probes and produce the same amplicon during PCR amplification. Because, in this example, both probes have different reporter dyes the PCR experiment was performed in the same well in dual color mode. To demonstrate the sufficiency of the PCR performance (even with different sample concentrations) the PCR was performed with two different dilutions of cDNA as target (in technical duplicates of each concentration). The difference of example 3 compared to example 1 is the PCR mode mono color to dual color (the parameter in example 3 is 18S).

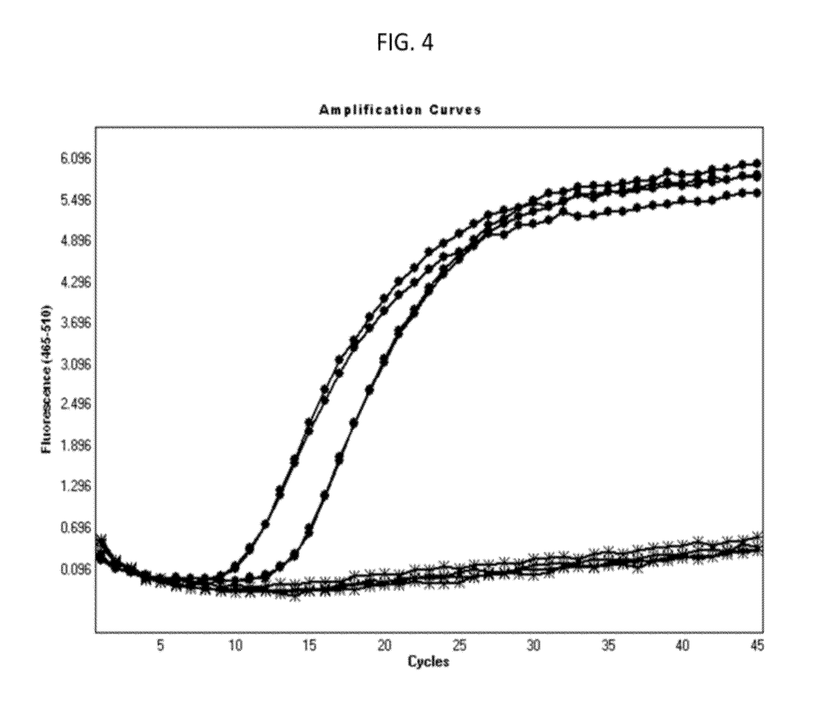

With reference to FIG. 4, the PCR reactions with a probe having a full match sequence to the target sequence (exemplified as first probe 110 in FIG. 1) show a sigmoidal amplification curve in the corresponding fluorescence channel and therefore a positive Cp call while the probe having a mis match to the target sequence (exemplified as second probe 120 in FIG. 1) show no sigmoidal amplification curve in the corresponding fluorescence channel and a negative Cp call. With reference to FIG. 4, data from a dual color assay (utilizing parameter 18S) according to the instant disclosure are shown. The circle style points illustrate a positive Cp call and a sigmoidal amplification curve (generated from a full match probe) at different sample concentrations. The curves having star style points (along the bottom) illustrate negative (no Cp) calls and no sigmoidal amplification curve (generated from a mismatch probe) at different sample concentrations. It should be noted (although it is clear from the specifications below) that signal from the full match probe is detected in a fluorescent channel for LC Yellow 555 and that signal from the mismatch probe is detected in the fluorescent channel for FAM.

The present experiment was carried out as illustrated in FIG. 1, according to the following specifications:

Dual Color PCR Assay with Parameter 18S

General probe structures:
5′-D-L-L-L-L-L-X-X-3′
LC Yellow 555 full match probe sequence:
5′-Y-t-T* [symbol] *T*T*G*-(AGCT)*(AGCT)*-Z-3′
FAM mismatch probe sequence:
5′-X-t-T* [symbol] *T*T*G*-(AGCT)*(AGCT)*-Z-3′

whereas X=FAM,
Y=LC Yellow 555 (LightCycler® Yellow 555), t=dT
T*, A*, C* or G*=LNA T, A, C or G
(ACCT)*=wobble bases of LNA T, A, C and G (random position)
Z=BHQ2-Quencher (Black Hole Quencher 2)
Bold and italic fonts indicate discriminating position.

(SEQ ID NO: 1)

| | |
|---|---|
| Primer forward sequence: | gacggaccagagcgaaag |

(SEQ ID NO: 2)

| | |
|---|---|
| Primer reverse sequence: | cgtcttcgaacctccgact |

PCR Cycler: LightCycler® 480 Instrument, 384-well block (Roche Applied Science, Cat. No.: 04 545 885 001)
PCR Reagents: LightCycler® 480 Probes Master (Roche Applied Science, Cat. No.: 04 707 494 001); LightCycler® 480 Multiwell Plate, 384 (Roche Applied Science, Cat. No.: 04 729 749 001)
Sample material: cDNA, reverse transcribed from RNA (Takara/Clontech, Cat. No.: 636538) with Transcriptor First Strand cDNA Synthesis Kit (Roche Applied Science, Cat. No.: 04 897 030 001). The cDNA synthesis step was performed as described in the pack insert of the corresponding kit.
Sample concentration: 50 ng and 5 ng per well in 10 μl final PCR volume

PCR Protocol for Example 3:

### FAM-Kanal:453:510

| | ° C. | Time | | RR(° C./s)(96) | Aq. Mode | Cycles |
|---|---|---|---|---|---|---|
| Preinkubation | 95 | 10 | min. | 4.8 (4, 4) | — | — |
| Amplifikation | 95 | 10 | s | 4.8 (4, 4) | none | 30 |
| denat. | | | | | | |
| anneal. | 37 | 30 | s | 2.5 (2, 2) | none | — |
| elong. | 50 | 30 | s | 4.8 (4, 4) | Single | — |
| Cooling | 40 | 30 | s | 2.2 (2, 5) | none | — |

All publications, patents and applications are hereby incorporated by reference in their entirety to the same extent as if each such reference was specifically and individually indicated to be incorporated by reference in its entirety.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within the known or customary practice in the art to which this disclosure pertains.

What is claimed is:

1. A composition comprising:
a first probe having a 5′ end opposite a 3′ end and at least eight nucleotides, the at least eight nucleotides comprising at least one DNA nucleotide and at least five locked nucleic acid nucleotides and a first discriminating position; and
a second probe having a 5′ end opposite a 3′ end and a same number of nucleotides as the first probe, the nucleotides of the second probe comprising a same number of DNA nucleotides and locked nucleic acid nucleotides as the first probe and a second discriminating position located at a position corresponding to the first discriminating position in the first probe,
wherein the nucleotides of the first and second probes comprise one of an adenine nucleobase, a cytosine nucleobase, a guanine nucleobase, a thymine nucleobase, a uracil nucleobase, and a methyl cytosine nucleobase, and wherein the first and second probes comprise differing nucleobases at the first and second discriminating positions and a same nucleobase at all other nucleotide positions.

2. The composition of claim 1 wherein the first and second probes have only eight nucleotides, the eight nucleotides comprising seven locked nucleic acid nucleotides and one DNA nucleotide, the one DNA nucleotide being located at one of a first nucleotide position and a second nucleotide position from the 5′ end of the first and second probes, and wherein the first and second discriminating positions comprise one of a third, a fourth, and a fifth nucleotide position from the 5′ end of the first and second probes.

3. The composition of claim 1, wherein the first probe incudes a first marker and the second probe includes a second marker.

4. The composition of claim 3, wherein the first marker and the second marker comprise fluorophores having a same excitation spectrum and a same emission spectrum.

5. The composition of claim 3, wherein the first probe incudes a quencher and the second probe includes a same quencher.

6. A composition comprising:
a first set of probes, each probe of the first set having a 5′ end opposite a 3′ end and eight nucleotides, the nucleotides of each probe comprising at least one DNA nucleotide, at least five locked nucleic acid nucleotides, and a first discriminatory position, at least one locked nucleic acid nucleotide being a random locked nucleic acid nucleotide; and
a second set of probes, each probe of the second set having a 5′ end opposite a 3′ end and eight nucleotides, each probe of the second set comprising a corresponding number of DNA nucleotides, locked nucleic acid nucleotides, and random locked nucleic acid nucleotides as a probe in the first set, and each probe of the second set having a second discriminating position located at a same nucleotide location as a first discriminating position of a probe in the first set,
wherein all probes of the first and second sets comprise a same nucleobase sequences with the exception of
(i) the nucleobase at the random locked nucleic acid nucleotides; and
(ii) the nucleobase at the first and second discriminating positions, wherein the nucleobase of the second discriminating position differs from the nucleobase of the first discriminating position at the same nucleotide location, and
wherein the at least one random locked nucleic acid nucleotide of each probe of the second set comprises a same nucleobase located at a same nucleotide location of the at least one random locked nucleic acid nucleotide of a probe of the first set, the nucleobase of the random locked nucleic acid nucleotide selected from one of adenine, cytosine, guanine, and thymine, and wherein any possible nucleobase sequence resulting from nucleobase variations at the one or more random locked nucleic acid nucleobase position(s) is represented by at least one probe in both the first and second set of probes.

7. The composition of claim 6, wherein the first and second discriminating positions are located at one of positions 2, 3, 4, 5, 6, and 7 from the 5′ end of each probe.

8. The composition of claim 6, wherein the nucleotide at position 1 from the 5′ end of each probe is a DNA nucleotide.

9. The composition of claim 6, wherein each probe consists of one of: one DNA nucleotide, seven locked nucleic acid nucleotides, and a marker; two DNA nucleotides, six locked nucleic acid nucleotides, and a marker; and three DNA nucleotides, five locked nucleic acid nucleotides, and a marker.

10. The composition of claim 9, wherein each probe further consists of a quencher.

11. The composition of claim 6, wherein each probe has one of: only one random locked nucleic acid nucleotide located at one of nucleotide positions 5, 6, 7 and 8 from the 5′ end of each probe; only two random locked nucleic acid nucleotides located at two of nucleotide positions 5, 6, 7 and 8; only three random locked nucleic acid nucleotides located at three of nucleotide positions 5, 6, 7, and 8.

12. The composition of claim 6, wherein each probe comprises the general structure 5′-D-L-L-L-L-X-X-X-3′, wherein D is a DNA nucleotide, each L is a LNA nucleotide and each X is a LNA random position.

13. The composition of claim 6, wherein each probe comprises the general structure 5′-DL L L L L X X-3′, wherein D is a DNA nucleotide, each L is a LNA nucleotide and each X is a LNA random position.

14. The composition of claim 6, wherein the probes of first set of probes are labeled with a first marker and the probes of the second set of probes are labeled with a second marker, the first marker being different from the second marker.

15. The composition of claim 14, wherein the probes of the first and second set of probes are also labeled with a quencher.

16. A method of determining a genotype at a locus of interest in a sample comprising genetic material, the method comprising the steps of:
contacting the genetic material with a first probe and a second probe; and
detecting the binding of one of the first and second probe to the genetic material, thereby determining the genotype at the locus,
wherein, the first and second probes each have a 5′ end opposite a 3′ end and eight nucleotides comprising at least one DNA nucleotide and at least five locked nucleic acid nucleotides, the nucleotides of the first probe comprising a first discriminating position and the nucleotides of the second probe comprising a second discriminating position at a same nucleotide location in the second probe as the first discriminating position in the first probe, the first discriminating position comprising a different nucleobase than the second discriminating position, wherein the nucleobases at the other nucleotides of the first and second probes being the same.

17. The method of claim 16, wherein the locus is a single nucleotide.

18. The method of claim 16 further comprising the steps of:
performing an amplifying step including contacting the genetic material with a first primer and a second primer, the amplifying step producing an amplification product including the locus of interest,
performing a hybridizing step comprising contacting the amplification product with the first probe and the second probe; and
detecting the hybridizing of one of the first and second probes to the genetic material, thereby determining the genotype at the locus.

19. The method of claim 16, wherein the step of detecting comprises measuring presence or absence of fluorescence of at least one of fluorescein, LC-Yellow 555, FAM, VIC, HEX, Rhodamine B, Rhodamine 6G, LC-Red 610, LC-Red 640, LC-Red 670, LC-Red 705, Cy3, Cy3.5, Cy5, and Cy5.5, and wherein the sample is one of a body fluid, a blood sample, a urine sample, serum, mucosa, sputum feces, epidermal sample, skin sample, cheek swab, sperm, amniotic fluid, cultured cells and bone marrow sample.

20. The method of claim 16, wherein the step of detecting comprises a real-time detection assay.
